(19) United States
(12) Patent Application Publication
(10) Pub. No.: US 2011/0002934 A1
(43) Pub. Date: Jan. 6, 2011

(54) USES OF ANTI-CD40 ANTIBODIES

(75) Inventors: Mohammad Luqman, Emeryville, CA (US); Sherna Budhabhatti, Danville, CA (US); Yongyu Wang, Morristown, NJ (US); Seema Kantak, Pacifica, CA (US); Ssucheng J. Hsu, Pinole, CA (US); Amer M. Mirza, San Francisco, CA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: Novartis AG; XOMA Technology, Ltd

(21) Appl. No.: 12/741,161

(22) PCT Filed: Nov. 7, 2008
(86) PCT No.: PCT/US08/82826

§ 371 Date: Sep. 8, 2010

Related U.S. Application Data
(60) Provisional application No. 61/002,545, filed on Nov. 9, 2007.

(51) Int. Cl. A61K 39/395; C12N 5/0781; A61P 35/00; A61P 35/02

(52) U.S. Cl. 424/139.1; 424/173.1; 424/154.1; 435/372.2

(57) ABSTRACT

This invention relates to new uses of anti-CD40 antibodies in the treatment of diseases or conditions associated with neoplastic B-cell growth in particular use of anti-CD40 antibodies in combination with cyclophosphamide, doxorubicin, vincristine and prednisone (CHOP). The invention is particularly useful for the treatment of patients who have previously been administered (i) CHOP, (ii) the chimeric anti-CD20 monoclonal antibody rituximab, or (iii) combination therapy with CHOP and rituximab.

### FIELD OF THE INVENTION

This invention relates to new uses of anti-CD40 antibodies in the treatment of diseases or conditions associated with neoplastic B-cell growth. The invention is particularly useful for the treatment of patients who have previously been administered (i) CHOP, (ii) the chimeric anti-CD20 monoclonal antibody rituximab, or (iii) combination therapy with CHOP and rituximab.

### BACKGROUND OF THE INVENTION

CD40 is a 50-55 kDa cell-surface antigen present on the surface of both normal and neoplastic human B-cells. Malignant B-cells from tumors of B-cell lineage express CD40 and appear to depend on CD40 signaling for survival and proliferation. Transformed cells from patients with low- and high-grade B-cell lymphomas, B-cell acute lymphoblastic leukemia, multiple myeloma, chronic lymphocytic leukemia, and Hodgkin's disease express CD40. CD40 expression is also detected in acute myeloblastic leukemia and 50% of AIDS-related lymphomas.

Anti-CD40 antibodies and uses thereof have been disclosed, e.g., in co-owned international patent applications published as WO 2005/044294, WO 2005/044304, WO 2005/044305, WO 2005/044306, WO 2005/044307, WO 2005/044854, WO 2005/044855, WO 2006/073443, WO 2006/125117, WO 2006/125143, WO 2007/053661 and WO 2007/053767. Those applications specifically disclose a human IgG₁ anti-CD40 monoclonal antibody, designated as CHIR-12.12 therein (but now known as HCD122), generated by immunization of transgenic mice bearing the human IgG₁ heavy chain locus and the human κ light chain locus (XenoMouse® technology; Abgenix, California). Those applications also disclose use of anti-CD40 antibodies, such as HCD122, for the treatment of diseases or conditions associated with neoplastic B-cell growth.

Although any one therapeutic agent may provide a benefit to the patient, further methods are needed to reduce toxicity and to improve treatment outcomes. In addition, diseases or conditions can often become refractory to treatment with single-agent therapy, either as a result of initial resistance or resistance that develops during therapy. Consequently, any discovery of a combination therapy that can improve treatment relative to single-agent therapy is of great interest.

### BRIEF DESCRIPTION OF THE FIGURES

FIG. 1 illustrates the results of an investigation into the anti-tumour activity provided by different treatments in the RL DLBCL xenograft model (see Example 1).

FIG. 2 illustrates the results of an investigation into the effects of CD40L and HCD122 on CHOP cytotoxicity on SU-DHL-4 cells.

FIG. 3 illustrates the results of an investigation into the effects of CD40L and HCD122 on NFkB signalling in RL and SU-DHL-4 cell lines.

FIG. 4 illustrates the results of an investigation into the effects of CD40L and HCD122 on expression of certain cell-surface adhesion molecules in RL cells.

FIG. 5 illustrates the results of an investigation into the effects of CD40L and HCD122 on expression of certain cell-surface adhesion molecules in SU-DHL-4 cells.

FIG. 6 illustrates the results of an investigation into the effects of CD40L and HCD122 on the in vitro aggregation of SU-DHL-4 cells.

### DETAILED DESCRIPTION OF THE INVENTION

The invention provides methods for treating human patients for diseases or conditions associated with neoplastic B-cell growth. The methods involve combination therapy with (i) an anti-CD40 antibody and (ii) cyclophosphamide, doxorubicin, vincristine and prednisone (CHOP). The inventors have discovered that administering these two known therapies in combination results in unexpectedly potent therapeutic efficacy in vivo. The inventors have found that the combined effect of these two therapies can be greater than the sum of the individual effects of each therapy, i.e., that the combination of an anti-CD40 antibody (such as HCD122) with CHOP can provide a synergistic therapeutic effect. Without wishing to be bound by the theory, the inventors believe that this unexpectedly potent therapeutic efficacy results from the ability of anti-CD40 antibodies to sensitize B-cells to CHOP cytotoxicity by down-regulating NF-kB activation and/or by inhibiting CD40L-induced expression of adhesion molecules.

The invention provides a method for treating a human patient for a disease or condition associated with neoplastic B-cell growth, said method comprising administering to said patient cyclophosphamide, doxorubicin, vincristine and prednisone (CHOP) in combination with an anti-CD40 antibody.

In some embodiments, the anti-CD40 antibody (herein “the antibody therapy”) and the cyclophosphamide, doxorubicin, vincristine and prednisone (CHOP; herein “the chemotherapy”) are administered to the patient at the same time. In these embodiments, the antibody therapy may be administered to the patient at exactly the same time as the chemotherapy (i.e., the two therapies are administered simultaneously). Alternatively, the antibody therapy may be administered to the patient at approximately the same time as the chemotherapy (i.e., the two therapies are not administered at precisely the same time), e.g., during the same visit to a physician or other healthcare professional.

In other embodiments, the antibody therapy and the chemotherapy are not administered to the patient at the same time, but are administered sequentially (consecutively) in either order. In these embodiments, the methods of the invention may comprise administering a first cycle of the chemotherapy to the patient before a first dose of the anti-CD40 antibody is administered to the patient. Alternatively, the methods may comprise administering a first cycle of the chemotherapy to the patient after a first dose of the anti-CD40 antibody is administered to the patient. In embodiments where the antibody therapy and the chemotherapy are administered sequentially, the therapies may be administered in such a way that both therapies exert a therapeutic effect on the patient at the same time (i.e., the periods in which each therapy is effective may overlap) although this is not essential.

The invention therefore provides a method for treating a human patient for a disease or condition associated with neoplastic B-cell growth, said method comprising administering to the patient an anti-CD40 antibody before, during, or after administering one or more of cyclophosphamide, doxorubicin, vincristine and prednisone. References herein to use of one or more of cyclophosphamide, doxorubicin, vincristine and prednisone are references to use of one or more, two or more, three or more, or all four, of cyclophosphamide, doxorubicin, vincristine and prednisone.

In embodiments where a first cycle of the chemotherapy is administered to the patient before a first dose of the anti-CD40 antibody, a first cycle of chemotherapy may be administered from about one week to about one year, from about one week to about ten months, from about one week to about eight months, from about one week to about six months, from about one week to about four months, from about one week to about two months, from about one week to about one month, from about one week to about three weeks, from about one week to about two weeks, or about one week, before the first dose of an anti-CD40 antibody is administered to the patient. In other words, the antibody therapy may be administered from about one week to about one year, from about one week to about ten months, from about one week to about eight months, from about one week to about six months, from about one week to about four months, from about one week to about two months, or from about one week to about one month, from about one week to about three weeks, from about one week to about two weeks, or about one week, after the first cycle of chemotherapy.

In embodiments where a first cycle of the chemotherapy is administered to the patient after a first dose of the anti-CD40 antibody, a first cycle of chemotherapy may be administered from about one week to about one year, from about one week to about ten months, from about one week to about eight months, from about one week to about six months, from about one week to about four months, from about one week to about two months, from about one week to about one month, from about one week to about three weeks, from about one week to about two weeks, or about one week, after the first dose of an anti-CD40 antibody is administered to the patient. In other words, the antibody therapy may be administered from about one week to about one year, from about one week to about ten months, from about one week to about eight months, from about one week to about six months, from about one week to about four months, from about one week to about two months, or from about one week to about one month, from about one week to about three weeks, from about one week to about two weeks, or about one week, before the first cycle of chemotherapy.

When the therapies are administered at the same time, they may be administered as a single pharmaceutical formulation or as two or more separate pharmaceutical formulations. When the therapies are not administered at the same time, they are administered as two or more separate pharmaceutical formulations.

When two or more separate pharmaceutical formulations are used, any suitable combination of the antibody therapy and the chemotherapy may be used. For example, one pharmaceutical formulation might contain the antibody therapy, whilst other pharmaceutical formulation(s) contain the chemotherapeutic agents cyclophosphamide, doxorubicin, vincristine and prednisone. Alternatively, one pharmaceutical formulation might contain the antibody therapy and one or more of the chemotherapeutic agents, whilst other pharmaceutical formulation(s) contain the other chemotherapeutic agent(s). In embodiments where a pharmaceutical formulation contains the antibody therapy and one or more of the chemotherapeutic agents, this pharmaceutical formulation may be obtained by a method comprising the steps of (i) obtaining a lyophilized anti-CD40 antibody composition, (ii) obtaining a composition comprising one or more of the chemotherapeutic agents in a sterile diluent, and (iii) reconstituting the lyophilized antibody composition using the composition comprising one or more of the chemotherapeutic agents.

The invention therefore provides a pharmaceutical composition comprising (i) one or more of cyclophosphamide, doxorubicin, vincristine and prednisone, (ii) an anti-CD40 antibody, and (iii) a pharmaceutically acceptable carrier or excipient.

The invention also provides the use of (i) one or more of cyclophosphamide, doxorubicin, vincristine and prednisone and (ii) an anti-CD40 antibody, in the manufacture of a medicament for treating a human patient for a disease or condition associated with neoplastic B-cell growth. In other embodiments, the invention provides the use of (i) one or more of cyclophosphamide, doxorubicin, vincristine and prednisone and (ii) an anti-CD40 antibody, in the manufacture of at least two separate medicaments (two, three, four or five medicaments) for treating a human patient for a disease or condition associated with neoplastic B-cell growth by combination therapy. The cyclophosphamide, vincristine, prednisone, doxorubicin and anti-CD40 antibody may be used in the manufacture of at least three, at least four, or five separate medicaments.

The invention also provides a kit for treating a human patient for a disease or condition associated with neoplastic B-cell growth, said kit comprising (i) one or more of cyclophosphamide, doxorubicin, vincristine and prednisone, and (ii) an anti-CD40 antibody. The kit may further comprise one or more devices for administering the combination therapy to a human patient, such as one or more of (i) a sterile needle and syringe, (ii) a sterile container (e.g., a glass bottle, plastic bottle or plastic bag) and drip chamber, (iii) a sterile tube with a regulating clamp, and (iv) a catheter.

The invention provides a method for treating a human patient for a disease or condition associated with neoplastic B-cell growth, said method comprising administering to said patient one or more of cyclophosphamide, doxorubicin, vincristine and prednisone, wherein the patient has been pre-treated with an anti-CD40 antibody. The invention also provides a method for treating a human patient for a disease or condition associated with neoplastic B-cell growth, said method comprising administering to said patient an anti-CD40 antibody, wherein the patient has been pre-treated with one or more of cyclophosphamide, doxorubicin, vincristine and prednisone.

The invention further provides the use of an anti-CD40 antibody in the manufacture of a medicament for treating a human patient for a disease or condition associated with neoplastic B-cell growth, wherein said human patient has been pre-treated with one or more of cyclophosphamide, doxorubicin, vincristine and prednisone. The invention also provides the use of one or more of cyclophosphamide, doxorubicin, vincristine and prednisone in the manufacture of a medicament for treating a human patient for a disease or condition associated with neoplastic B-cell growth, wherein said human patient has been pre-treated with an anti-CD40 antibody.

By “pre-treated” or “pre-treatment” is intended the subject has received one or more doses of a first therapy prior to a second therapy. “Pre-treated” or “pre-treatment” includes patients that have been treated with a first therapy within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or within 1 day prior to initiation of treatment with a second therapy. In the combination methods of the invention, “pre-treated” or “pre-treatment” thus includes patients that have been treated with an anti-CD40 antibody within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or within 1 day prior to initiation of treatment with the chemotherapy. In the combination methods of the invention, “pre-treated” or “pre-treatment” also includes patients that have been treated with the chemotherapy within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or within 1 day, prior to initiation of treatment with an anti-CD40 antibody.

Patients who have been pre-treated with an anti-CD40 antibody can be distinguished from other patients, e.g., by consulting patients' medical records or carrying out suitable in vitro test(s). Patients who have been pre-treated with one or more of cyclophosphamide, doxorubicin, vincristine and prednisone can be distinguished from other patients, e.g., by consulting patients' medical records or carrying out suitable in vitro test(s).

The invention also provides the use of an anti-CD40 antibody in the manufacture of a medicament for treating a human patient for a disease or condition associated with neoplastic B-cell growth, wherein the medicament is administered prior to cyclophosphamide, doxorubicin, vincristine or prednisone. In alternative embodiments, the invention provides the use of an anti-CD40 antibody in the manufacture of a medicament for treating a human patient for a disease or condition associated with neoplastic B-cell growth, wherein the medicament is administered subsequent to cyclophosphamide, doxorubicin, vincristine or prednisone. The invention also provides the use of one or more of cyclophosphamide, doxorubicin, vincristine and prednisone in the manufacture of a medicament for treating a human patient for a disease or condition associated with neoplastic B-cell growth, wherein the medicament is administered prior to an anti-CD40 antibody. In alternative embodiments, the invention provides the use of one or more of cyclophosphamide, doxorubicin, vincristine and prednisone in the manufacture of a medicament for treating a human patient for a disease or condition associated with neoplastic B-cell growth, wherein the medicament is administered subsequent to an anti-CD40 antibody.

The invention also provides an anti-CD40 antibody and one or more of cyclophosphamide, doxorubicin, vincristine and prednisone, for simultaneous, separate or sequential use in treating a human patient for a disease or condition associated with neoplastic B-cell growth by combination therapy. The invention also provides the use of an anti-CD40 antibody in the manufacture of a medicament for simultaneous or sequential use in combination with one or more of cyclophosphamide, doxorubicin, vincristine and prednisone for treating a human patient for a disease or condition associated with neoplastic B-cell growth. The invention also provides the use of one or more of cyclophosphamide, doxorubicin, vincristine and prednisone in the manufacture of a medicament for simultaneous or sequential use in combination with an anti-CD40 antibody for treating a human patient for a disease or condition associated with neoplastic B-cell growth.

The methods of the invention may comprise administering a dose of an anti-CD40 antibody at any time during a first or subsequent cycle of the chemotherapy. Alternatively, the methods of the invention may comprise administering a dose of an anti-CD40 antibody between cycles of chemotherapy.

As noted above, the inventors have found that combination therapy with an anti-CD40 antibody and CHOP can provide a synergistic therapeutic effect. Accordingly, in some embodiments of the methods, uses, compositions and kits disclosed herein, the combination therapy provides a synergistic improvement in therapeutic efficacy relative to the individual therapeutic agents when administered alone. The term “synergy” is used to describe a combined effect of two or more active agents that is greater than the sum of the individual effects of each respective active agent. Thus, where the combined effect of two or more agents results in “synergistic inhibition” of an activity or process, for example, tumor growth, it is intended that the inhibition of the activity or process is greater than the sum of the inhibitory effects of each respective active agent. The term “synergistic therapeutic effect” therefore refers to a therapeutic effect observed with a combination of two or more therapies wherein the therapeutic effect (as measured by any of a number of parameters, e.g., tumour growth delay as in Example 1 herein) is greater than the sum of the individual therapeutic effects observed with the respective individual therapies.

As noted above, the inventors believe that the unexpectedly potent therapeutic efficacy provided by the combination therapy of the invention results from the ability of anti-CD40 antibodies to sensitize neoplastic B-cells to CHOP cytotoxicity by down-regulating NF-kB activation and/or by inhibiting CD40L-induced expression of adhesion molecules (see Examples 2-4 herein). The examples herein demonstrate that signalling via CD40 might contribute to the development of B-cell resistance to CHOP cytotoxicity, and that this resistance might be prevented or reduced by using an antagonistic anti-CD40 antibody (such as HCD122) to reduce CD40 signalling. The examples herein further demonstrate that expression of cell-surface adhesion molecules on B-cells induced by CD40 signalling might contribute to the development of B-cell resistance to CHOP cytotoxicity, by allowing B-cells to aggregate and interact with their microenvironment. The examples suggest that expression of cell-surface adhesion molecules on B-cells might be prevented or reduced by using an antagonistic anti-CD40 antibody (such as HCD 122).

Accordingly, the invention provides the use of an anti-CD40 antibody to prevent or reduce resistance to CHOP cytotoxicity in neoplastic human B-cells (i.e., to sensitize neoplastic B-cells to CHOP cytotoxicity). The invention also provides a method for preventing or reducing resistance to CHOP cytotoxicity in neoplastic human B-cells (i.e., sensitising neoplastic B-cells to CHOP cytotoxicity), comprising the step of contacting in vitro one or more neoplastic human B-cells with an anti-CD40 antibody.

The invention further provides a method for preventing or reducing B-cell resistance to CHOP cytotoxicity in a human patient, comprising the step of administering an anti-CD40 antibody to the patient. The invention also provides a method for treating a human patient for a disease or condition associated with neoplastic B-cell growth, said method comprising a step of reducing B-cell resistance to CHOP cytotoxicity in said patient (i.e., sensitising the patient's neoplastic B-cells to CHOP cytotoxicity) by administering an anti-CD40 antibody to the patient.

The invention also provides an anti-CD40 antibody, for preventing or reducing resistance to CHOP cytotoxicity in neoplastic human B-cells in vitro (i.e., for sensitising neoplastic B-cells to CHOP cytotoxicity) or in a human patient in vivo (i.e., sensitising the patient's neoplastic B-cells to CHOP cytotoxicity). The invention also provides the use of an anti-CD40 antibody in the manufacture of a medicament for preventing or reducing B-cell resistance to CHOP cytotoxicity in a human patient (i.e., sensitising the patient's neoplastic B-cells to CHOP cytotoxicity).

Preferably, the anti-CD40 antibody used in these embodiments down-regulates NF-kB activation. In particular, the antibody may down-regulate the NF-kB activation in B-cells that is induced by CD40 signalling and which contributes to the development of B-cell resistance to CHOP cytotoxicity.

Preferably, the anti-CD40 antibody used in these embodiments is an antibody that inhibits the expression of one or more cell-surface adhesion molecules on B-cells. In particular, the antibody may inhibit the expression of one or more cell-surface adhesion molecules on B-cells that is induced by CD40 signalling and which contribute(s) to the development of B-cell resistance to CHOP cytotoxicity. In some embodiments, the anti-CD40 antibody inhibits CD40-L induced expression of one or more of CD54, CD80, CD86 and CD95 (or two or more, three or more, or all four, of CD54, CD80, CD86 and CD95).

The compositions, uses and kits of the invention may therefore use an anti-CD40 antibody that is capable of down-regulating NF-kB activation and/or which is capable of inhibiting the expression of one or more cell-surface adhesion molecules on B-cells.

A summary of standard techniques and procedures which may be employed in order to utilize the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification. The practice of the present invention will employ, unless otherwise indicated, conventional techniques of molecular biology, microbiology, recombinant DNA technology and immunology, which are within the skill of those working in the art. Such techniques are explained fully in the literature.

The invention involves the use of anti-CD40 antibodies for the treatment of human patients having diseases or conditions associated with neoplastic B-cell growth. By “CD40”, “CD40 antigen”, or “CD40 receptor” is intended the 50-55 kDa transmembrane glycoprotein of the tumor necrosis factor (TNF) receptor family (see, for example, U.S. Pat. Nos. 5,674,492 and 4,708,871; Stamenkovic et al. (1989) EMBO 8:1403; Clark (1990) Tissue Antigens 36:33; Barclay et al. (1997) The Leucocyte Antigen Facts Book (2d ed.; Academic Press, San Diego)). Two isoforms of human CD40, encoded by alternatively spliced transcript variants of this gene, have been identified. The first isoform (also known as the “long isoform” or “isoform 1”) is expressed as a 277-amino-acid precursor polypeptide (SEQ ID NO:9; first reported as GenBank Accession No. CAA43045, and identified as isoform 1 in GenBank Accession No. NP_{—}001241), encoded by SEQ ID NO:8 (see GenBank Accession Nos. X60592 and NM_{—}001250), which has a signal sequence represented by the first 19 residues. The second isoform (also known as the “short isoform” or “isoform 2”) is expressed as a 203-amino-acid precursor polypeptide (SEQ ID NO:7; GenBank Accession No. NP_{—}690593), encoded by SEQ ID NO:6 (GenBank Accession No. NM_{—}152854), which also has a signal sequence represented by the first 19 residues. The precursor polypeptides of these two isoforms of human CD40 share in common their first 165 residues (i.e., residues 1-165 of SEQ ID NO:7 and SEQ ID NO:9). The precursor polypeptide of the short isoform (shown in SEQ ID NO:7) is encoded by a transcript variant (SEQ ID NO:6) that lacks a coding segment, which leads to a translation frame shift; the resulting CD40 isoform contains a shorter and distinct C-terminus (residues 166-203 of SEQ ID NO:7) from that contained in the long isoform of CD40 (C-terminus shown in residues 166-277 of SEQ ID NO:9). For purposes of the present invention, the term “CD40,” or “CD40 antigen,” “CD40 cell surface antigen,” or “CD40 receptor” encompasses both the short and long isoforms of CD40.

By “CD40-expressing cells” herein is intended any normal or malignant cells that express detectable levels of the CD40 antigen. Methods for detecting CD40 antigen expression in cells are well known in the art and include, but are not limited to, PCR techniques, immunohistochemistry, flow cytometry, Western blot, ELISA, and the like. These methods allow for the detection of CD40 mRNA, CD40 antigen and cell-surface CD40 antigen. Preferably, the CD40-expressing cells are cells that express detectable levels of cell-surface CD40 antigen.

By “CD40 ligand” or “CD40L” is intended the 32-33 kDa transmembrane protein that also exists in two smaller biologically active soluble forms, 18 kDa and 31 kDa, respectively (Graf et al. (1995) Eur. J. Immunol. 25:1749-1754; Mazzei et al. (1995) J. Biol. Chem. 270:7025-7028; Pietravalle et al. (1996) J. Biol. Chem. 271:5965-5967). Human CD40L is also known as CD154 or gp39.

By “human patient” is intended a human who is afflicted with, at risk of developing or relapsing with, any disease or condition associated with neoplastic B-cell growth.

By “disease or condition associated with neoplastic B-cell growth” is intended any disease or condition (including pre-malignant conditions) involving uncontrolled growth of cells of B-cell lineage. Such diseases and conditions include, but are not limited to, acute lymphoblastic leukemia (ALL), acute myelogenous leukemia (AML), chronic myelogenous leukemia (CML), chronic lymphocytic leukemia (CLL), prolymphocytic leukemia (PLL), small lymphocytic leukemia (SLL), diffuse small lymphocytic leukemia (DSLL), diffuse large B-cell lymphoma (DLBCL), hairy cell leukemia, non-Hodgkin's lymphomas, Hodgkin's disease, Epstein-Barr Virus (EBV) induced lymphomas, myelomas such as multiple myeloma, Waldenstrom's macroglobulinemia, heavy chain disease, mucosal associated lymphoid tissue lymphoma, monocytoid B cell lymphoma, splenic lymphoma, lymphomatoid granulomatosis, intravascular lymphomatosis, immunoblastic lymphomas, AIDS-related lymphomas, and the like.

The methods of the invention find use in the treatment of subjects having non-Hodgkin's lymphomas related to abnormal B cell proliferation or accumulation. For purposes of the present invention, such lymphomas will be referred to according to the Working Formulation classification scheme, that is those B cell lymphomas categorized as low grade, intermediate grade, and high grade (see “The Non-Hodgkin's Lymphoma Pathologic Classification Project,” Cancer 49 (1982):2112-2135). Thus, low-grade B cell lymphomas include small lymphocytic, follicular small-cleaved cell, and follicular mixed small-cleaved and large cell lymphomas; intermediate-grade lymphomas include follicular large cell, diffuse small cleaved cell, diffuse mixed small and large cell, and diffuse large cell lymphomas; and high-grade lymphomas include large cell immunoblastic, lymphoblastic, and small non-cleaved cell lymphomas of the Burkitt's and non-Burkitt's type. The methods of the invention can be used to treat low-, intermediate-, and high-grade B cell lymphomas.

The methods of the invention are useful in the therapeutic treatment of B cell lymphomas that are classified according to the Revised European and American Lymphoma Classification (REAL) system. Such B cell lymphomas include, but are not limited to, lymphomas classified as precursor B cell neoplasms, such as B lymphoblastic leukemia/lymphoma; peripheral B cell neoplasms, including B cell chronic lymphocytic leukemia/small lymphocytic lymphoma, lymphoplasmacytoid lymphoma/immunocytoma, mantle cell lymphoma (MCL), follicle center lymphoma (follicular) (including diffuse small cell, diffuse mixed small and large cell, and diffuse large cell lymphomas), marginal zone B-cell lymphoma (including extranodal, nodal, and splenic types, e.g., extranodal marginal zone B-cell lymphoma of mucosa-associated lymphoid tissue), plasmacytoma/20 myeloma, diffuse large cell B cell lymphoma of the subtype primary mediastinal (thymic), Burkitt's lymphoma, and Burkitt's like high-grade B cell lymphoma; and unclassifiable low-grade or high-grade B cell lymphomas.

In the methods of the invention, combination therapy is used to provide a positive therapeutic response with respect to a disease or condition. By “positive therapeutic response” is intended an improvement in the disease or condition, and/or an improvement in the symptoms associated with the disease or condition, as a result of the therapeutic activity of the combination therapy. That is, an anti-proliferative effect, the prevention of further tumor outgrowths, a reduction in tumor size, a reduction in the number of neoplastic cells, and/or a decrease in one or more symptoms associated with CD40-expressing cells can be observed. Thus, for example, a positive therapeutic response would refer to one or more of the following improvements in the disease: (1) a reduction in tumor size; (2) a reduction in the number of neoplastic cells; (3) an increase in neoplastic cell death; (4) inhibition of neoplastic cell survival; (4) inhibition (i.e., slowing to some extent, preferably halting) of tumor growth; (5) inhibition (i.e., slowing to some extent, preferably halting) of neoplastic cell infiltration into peripheral organs; (6) inhibition (i.e., slowing to some extent, preferably halting) of tumor metastasis; (7) the prevention of further tumor outgrowths; (8) an increased patient survival rate; and (9) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation. In addition to these positive therapeutic responses, the subject undergoing therapy may experience the beneficial effect of an improvement in the symptoms associated with the disease. Thus for B cell tumors, the subject may experience a decrease in the so-called B symptoms, i.e., night sweats, fever, weight loss, and/or urticaria. For pre-malignant conditions, therapy with an anti-CD40 therapeutic agent may block and/or prolong the time before development of a related malignant condition, for example, development of multiple myeloma in subjects suffering from monoclonal gammopathy of undetermined significance (MGUS).

An improvement in the disease may be characterized as a complete response. By “complete response” is intended an absence of clinically detectable disease with normalisation of any previously abnormal radiographic studies, bone marrow, and cerebrospinal fluid (CSF) or abnormal monoclonal protein in the case of myeloma. Such a response may persist for at least 4 to 8 weeks, or sometimes 6 to 8 weeks, following treatment according to the methods of the invention. Alternatively, an improvement in the disease may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

The methods and products of the invention involve use of therapeutically or prophylactically effective amounts of an anti-CD40 antibody and each of the four CHOP components. By “an effective amount” or “therapeutically or prophylactically effective amount” is intended an amount of antibody therapy or chemotherapy that, when administered as a part of a combination therapy, brings about a positive therapeutic response with respect to patient treatment. Suitable amounts are described in more detail elsewhere herein.

“Tumor” (or “tumour”), as used herein, refers to all neoplastic cell growth and proliferation, whether malignant or benign, and all pre-cancerous and cancerous cells and tissues. “Neoplastic”, as used herein, refers to any form of dysregulated or unregulated cell growth, whether malignant or benign, resulting in abnormal growth. Thus, “neoplastic cells” include malignant and benign cells having dysregulated or unregulated cell growth. The terms “cancer” and “cancerous” refer to or describe the physiological condition in mammals that is typically characterized by unregulated cell growth.

“Treatment” is herein defined as the application or administration of combination therapy to a patient, or application or administration of combination therapy to an isolated tissue from a patient, where the patient has a disease, a symptom of a disease, or a predisposition toward a disease, where the purpose is to cure, heal, alleviate, relieve, alter, remedy, ameliorate, improve, or affect the disease, the symptoms of the disease, or the predisposition toward the disease.

The methods of the invention are particularly useful for treating patients who have previously been administered other oncotherapeutic treatments. This includes patients who have been administered another oncotherapeutic treatment at any time prior to initiation of the combination treatment according to the invention, e.g., within 15 years, within 14 years, within 13 years, within 12 years, within 11 years, within 10 years, within 9 years, within 8 years, within 7 years, within 6 years, within 5 years, within 4 years, within 3 years, within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or within 1 day, prior to initiation of the combination treatment according to the invention.

In particular, the combination therapy of the invention may be useful for treating a human patient who has previously been administered (i) CHOP alone, (ii) an anti-CD40 antibody (such as HCD122) alone, (iii) an anti-CD20 antibody (such as the chimeric anti-CD20 antibody rituximab) alone, or (iv) combination therapy with CHOP and an anti-CD20 antibody (such as rituximab, wherein the combination therapy is commonly termed R-CHOP).

The invention may be particularly useful for treating diseases or conditions that are refractory to therapy with other oncotherapeutic treatments. The invention may therefore be useful in treating diseases or conditions that are refractory to therapy with (i) CHOP alone, (ii) an anti-CD40 antibody (such as HCD122) alone, (iii) an anti-CD20 antibody (such as rituximab) alone, or (iv) combination therapy with CHOP and an anti-CD20 antibody (R-CHOP). By “refractory” is intended the particular disease or condition is resistant to, or non-responsive to, therapy with a particular oncotherapeutic agent. A disease or condition can be refractory to therapy with a particular therapeutic agent either from the onset of treatment with the particular therapeutic agent (i.e., non-responsive to initial exposure to the therapeutic agent), or as a result of developing resistance to the therapeutic agent, either over the course of a first treatment period with the therapeutic agent or during a subsequent treatment period with the therapeutic agent. The invention therefore provides methods, compositions, uses and kits for treating a human patient for a disease or condition associated with neoplastic B-cell growth, wherein said disease or condition is refractory to an oncotherapeutic treatment other than the combination therapy of the invention. The term “oncotherapeutic” is intended to mean any treatment for disease or condition, such as chemotherapy, antibody therapy, surgery, radiation therapy, and combinations thereof.

The invention may also be particularly useful for treating patients who have relapsed after therapy with other oncotherapeutic treatments. The invention may therefore be useful in treating patients who have relaped after therapy with (i) CHOP alone, (ii) an anti-CD40 antibody (such as HCD122) alone, (iii) an anti-CD20 antibody (such as rituximab) alone, or (iv) combination therapy with CHOP and an anti-CD20 antibody (R-CHOP). By “relapsed” is meant that the patient achieved a partial or complete response to a prior oncotherapeutic treatment, but has subsequently had a recurrence of the disease or condition. The invention therefore provides methods, compositions, uses and kits for treating a human patient for a disease or condition associated with neoplastic B-cell growth, wherein said patient has relapsed after therapy with an oncotherapeutic treatment other than the combination therapy of the invention.

The combination therapy of the invention addresses problems associated with therapy using rituximab (the IDEC-C2B8 monoclonal antibody (Biogen Idec or Genentech) commercially available under the tradename Rituxan®). Rituximab is a chimeric anti-CD20 monoclonal antibody containing human IgG1 and kappa constant regions with murine variable regions isolated from a murine anti-CD20 monoclonal antibody (Reff et al. (1994) Blood 83:435-445). The methods of the invention enable the treatment of patients having a disease or condition associated with CD40-expressing B-cells, which might otherwise have been treated with rituximab or by combination therapy with rituximab and chemotherapeutic agents (e.g., CHOP).

Accordingly, the invention also provides methods, compositions, uses and kits for treating a human patient for a disease or condition associated with neoplastic B-cell growth by combination therapy, wherein the patient has previously been administered the chimeric anti-CD20 antibody rituximab. The invention may be useful in treating diseases or conditions that are refractory to therapy with (i) rituximab alone, or (ii) combination therapy with CHOP and rituximab (R-CHOP). The invention may also be useful in treating patients who have relaped after therapy with (i) rituximab alone, or (ii) combination therapy with CHOP and rituximab (R-CHOP).

Patients who have been pre-treated with rituximab can be distinguished from other patients, e.g., by consulting patients' medical records or carrying out suitable in vitro test(s). For example, the number of circulating CD19⁺ B-cells is depleted in patients treated with rituximab, and numbers of circulating CD19⁺ B-cells can be monitored using suitable methods, e.g., FACS (McLaughlin et al. (1998) J. Clin. Oncol. 16(8):2825-2833; Maloney et al. (1997) Blood 90(6):2188-2195).

The methods of the invention involve the use of anti-CD40 antibodies. Natural antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains. The term “variable” refers to the fact that certain portions of the variable domains differ extensively in sequence among antibodies. The variable regions confer antigen-binding specificity. The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The “light chains” of antibodies from any vertebrate species can be assigned to one of two clearly distinct types, called kappa (κ) and lambda (λ), based on the amino acid sequences of their constant domains.

Depending on the amino acid sequence of the constant domain of their “heavy chains”, antibodies can be assigned to different classes. There are five major classes of human antibodies: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of antibodies are called alpha, delta, epsilon, gamma, and mu, respectively. The subunit structures and three-dimensional configurations of different classes of antibodies are well known. Different isotypes have different effector functions. For example, human IgG1 and IgG3 isotypes have ADCC (antibody dependent cell-mediated cytotoxicity) activity. IgG1 antibodies, in particular human IgG1 antibodies, are particularly useful in the methods of the invention.

“Human effector cells” are leukocytes that express one or more FcRs and perform effector functions. Preferably, the cells express at least FcγRIII and carry out antigen-dependent cell-mediated cyotoxicity (ADCC) effector function. Examples of human leukocytes that mediate ADCC include peripheral blood mononuclear cells (PBMC), natural killer (NK) cells, monocytes, macrophages, eosinophils, and neutrophils, with PBMCs and NK cells being preferred. Antibodies that have ADCC activity are typically of the IgG1 or IgG3 isotype. Note that in addition to isolating IgG1 and IgG3 antibodies, ADCC-mediating antibodies can be made by combining a variable region from a non-ADCC antibody with an IgG1 or IgG3 isotype constant region.

The terms “Fc receptor” or “FcR” are used to describe a receptor that binds to the Fc region of an antibody. The preferred FcR is a native-sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRI receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see Daeron (1997) Annu. Rev. Immunol. 15:203-234). FcRs are reviewed in Ravetch and Kinet (1991) Annu. Rev. Immunol. 9:457-492 (1991); Capel et al. (1994) Immunomethods 4:25-34; and de Haas et al. (1995) J. Lab. Clin. Med. 126:330-341. Other FcRs, including those to be identified in the future, are encompassed by the term “FcR” herein. The term also includes the neonatal receptor, FcRn, which is responsible for the transfer of maternal IgGs to the fetus (Guyer et al. (1976) J. Immunol. 117:587 and Kim et al. (1994)J. Immunol. 24:249 (1994)).

The term “antibody” is used herein in the broadest sense and covers fully assembled antibodies, antibody fragments which retain the ability to specifically bind to the CD40 antigen (e.g., Fab, F(ab′)₂, Fv, and other fragments), single chain antibodies (scFv), diabodies, bispecific antibodies, chimeric antibodies, humanized antibodies, fully human antibodies, and the like, and recombinant peptides comprising the foregoing. The term “antibody” covers both polyclonal and monoclonal antibodies.

As used herein “anti-CD40 antibody” encompasses any antibody that specifically recognizes the CD40 antigen. In some embodiments, anti-CD40 antibodies for use in the methods of the present invention, in particular monoclonal anti-CD40 antibodies, exhibit a strong single-site binding affinity for the CD40 antigen. Such monoclonal antibodies exhibit an affinity for CD40 (K_{D}) of at least 10⁻⁵ M, preferably at least 10⁻⁶ M, at least 10⁻⁷ M, at least 10⁻⁸ M, at least 10⁻⁹ M, at least 10⁻¹⁰ M, at least 10⁻¹¹ M or at least 10⁻¹² M, when measured using a standard assay such as Biacore™. Biacore analysis is known in the art and details are provided in the “BIAapplications handbook”.

By “specifically recognizes” or “specifically binds to” is intended that the anti-CD40 antibody binds to the CD40 antigen on the surface of human B-cells, but does not bind to a significant extent other antigens on the surface of human B-cells, such as the CD20 antigen.

The anti-CD40 antibodies for use in the methods of the present invention can be produced using any suitable antibody production method known to those of skill in the art.

The anti-CD40 antibody used in the methods of the present invention may be a monoclonal antibody. The term “monoclonal antibody” (and “mAb”) as used herein refers to an antibody obtained from a substantially homogeneous population of antibodies, i.e., the individual antibodies comprising the population are identical except for possible naturally occurring mutations that may be present in minor amounts. The term is not limited regarding the species of the antibody and does not require production of the antibody by any particular method. In contrast to polyclonal antibody preparations, which typically include different antibodies directed against different antigenic determinants (epitopes), each monoclonal antibody is directed against a single determinant (epitope) on the antigen.

The term “monoclonal” as originally used in relation to antibodies referred to antibodies produced by a single clonal line of immune cells, as opposed to “polyclonal” antibodies that, while all recognizing the same target protein, were produced by different B cells and would be directed to different epitopes on that protein. As used herein, the word “monoclonal” does not imply any particular cellular origin, but refers to any population of antibodies that all have the same amino acid sequence and recognize the same epitope in the same target protein. Thus a monoclonal antibody may be produced using any suitable protein synthesis system, including immune cells, non-immune cells, acellular systems, etc. This usage is usual in the field e.g., the product datasheets for the CDR-grafted humanized antibody Synagis™ expressed in a murine myeloma NSO cell line, the humanized antibody Herceptin™ expressed in a CHO cell line, and the phage-displayed antibody Humira™ expressed in a CHO cell line all refer to the products as monoclonal antibodies.

By “epitope” is intended the part of an antigenic molecule to which an antibody is produced and to which the antibody will bind. Epitopes can comprise linear amino acid residues (i.e., residues within the epitope are arranged sequentially in a linear fashion), non-linear amino acid residues (referred to herein as “non-linear epitopes”; these epitopes are not arranged sequentially), or both linear and non-linear amino acid residues.

Monoclonal antibodies may be made by the hybridoma method first described by Kohler et al. (1975) Nature 256:495, or may be made by recombinant DNA methods (see, e.g., U.S. Pat. No. 4,816,567). Monoclonal antibodies may also be isolated from antibody phage libraries generated using the techniques described in, for example, McCafferty et al. (1990) Nature 348:552-554 (1990) and U.S. Pat. No. 5,514,548. Clackson et al., (1991) Nature 352:624-628 and Marks et al. (1991) J. Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

Where anti-CD40 antibodies for use in the methods of the invention are to be prepared using recombinant DNA methods, the DNA encoding the monoclonal antibodies is readily isolated and sequenced using conventional procedures. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al. (1993) Curr. Opinion in Immunol. 5:256 and Phickthun (1992) Immunol. Revs. 130:151. Alternatively, antibody can be produced in a cell line such as a CHO cell line, as disclosed in U.S. Pat. Nos. 5,545,403; 5,545,405; and 5,998,144. Briefly the cell line is transfected with vectors capable of expressing a light chain and a heavy chain, respectively. By transfecting the two proteins on separate vectors, chimeric antibodies can be produced. Another advantage is the mammalian glycosylation of the antibody in CHO cells. CHO cells are a preferred source of recombinant antibodies for use in the combination therapy of the invention.

A “host cell,” as used herein, refers to a microorganism or a eukaryotic cell or cell line cultured as a unicellular entity that can be, or has been, used as a recipient for a recombinant vector or other transfer polynucleotides, and include the progeny of the original cell that has been transfected. It is understood that the progeny of a single cell may not necessarily be completely identical in morphology or in genomic or total DNA complement as the original parent, due to natural, accidental, or deliberate mutation.

Monoclonal antibodies to CD40 are known in the art. See, for example, the sections dedicated to B-cell antigen in McMichael, ed. (1987; 1989) Leukocyte Typing III and IV (Oxford University Press, New York); U.S. Pat. Nos. 5,674,492; 5,874,082; 5,677,165; 6,056,959; WO 00/63395; International Publication Nos. WO 02/28905 and WO 02/28904; Gordon et al. (1988) J. Immunol. 140:1425; Valle et al. (1989) Eur. J. Immunol. 19:1463; Clark et al. (1986) PNAS 83:4494; Paulie et al. (1989) J. Immunol. 142:590; Gordon et al. (1987) Eur. J. Immunol. 17:1535; Jabara et al. (1990) J. Exp. Med. 172:1861; Zhang et al. (1991) J. Immunol. 146:1836; Gascan et al. (1991) J. Immunol. 147:8; Banchereau et al. (1991) Clin. Immunol. Spectrum 3:8; and Banchereau et al. (1991) Science 251:70.

As noted above, the term antibody as used herein encompasses chimeric antibodies. By “chimeric” antibodies is intended antibodies that are most preferably derived using recombinant DNA techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to CD40. The non-human source can be any vertebrate source that can be used to generate antibodies to CD40 antigen. Such non-human sources include, but are not limited to, rodents (e.g., rabbit, rat, mouse, etc.; see, for example, U.S. Pat. No. 4,816,567) and non-human primates (e.g., Old World Monkey, Ape, etc.; see, for example, U.S. Pat. Nos. 5,750,105 and 5,756,096). The phrase “constant region” refers to the portion of the antibody molecule that confers effector functions. In previous work directed towards producing non-immunogenic antibodies for use in therapy of human disease, mouse constant regions were substituted by human constant regions. The constant regions of the subject humanized antibodies were derived from human antibodies. However, these antibodies can elicit an unwanted and potentially dangerous immune response in humans and there was a loss of affinity.

As noted above, the term antibody as used herein encompasses humanized antibodies. By “humanized” is intended forms of antibodies that contain minimal sequence derived from non-human antibody sequences. For the most part, humanized antibodies are human antibodies (recipient antibody) in which residues from a hypervariable region (also known as complementarity determining region or CDR) of the recipient are replaced by residues from a hypervariable region of a non-human species (donor antibody) such as mouse, rat, rabbit, or nonhuman primate having the desired specificity, affinity, and capacity. The phrase “complementarity determining region” refers to amino acid sequences which together define the binding affinity and specificity of the natural Fv region of a native antibody binding site. See, e.g., Chothia et al (1987) J. Mol. Biol. 196:901-917; Kabat et al (1991) U.S. Dept. of Health and Human Services, NIH Publication No. 91-3242).

Humanization can be performed following the method of Winter and co-workers (Jones et al. (1986) Nature 321:522-525; Riechmann et al. (1988) Nature 332:323-327; Verhoeyen et al. (1988) Science 239:1534-1536), by substituting rodent or mutant rodent CDRs or CDR sequences for the corresponding sequences of a human antibody. See also U.S. Pat. Nos. 5,225,539; 5,585,089; 5,693,761; 5,693,762; 5,859,205. In some instances, residues within the framework regions of one or more variable regions of the human antibody are replaced by corresponding non-human residues (see, for example, U.S. Pat. Nos. 5,585,089; 5,693,761; 5,693,762; and 6,180,370). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human antibody and all or substantially all of the framework regions are those of a human antibody sequence. The humanized antibody optionally also will comprise at least a portion of an antibody constant region (Fc), typically that of a human antibody. For further details see Jones et al. (1986) Nature 331:522-525; Riechmann et al. (1988) Nature 332:323-329; and Presta (1992) Curr. Op. Struct. Biol. 2:593-596. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

Humanized anti-CD40 antibodies can also be produced using the Human Engineering™ technology (Xoma Ltd., Berkeley, Calif.), which has been described as a method for reducing immunogenicity while maintaining binding activity of antibody molecules (e.g., see Studnicka et al. (1994) Protein Engineering 7:805-814 and U.S. Pat. No. 5,766,886).

Humanized anti-CD40 monoclonal antibodies include antibodies such as SGN-40 (Tai et al. (2004) Cancer Res. 64:2846-52; U.S. Pat. No. 6,838,261), which is the humanized form of the murine anti-CD40 antibody SGN-14 (Francisco et al. (2000) Cancer Res. 60:3225-31), and the antibodies disclosed in U.S. Patent Application Publication No. 2004/0120948.

The present invention can also be practiced using xenogeneic or modified antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. Nos. 5,877,397 and 5,939,598.

Thus, in some embodiments, fully human antibodies to CD40, for example, are obtained by immunizing transgenic mice. One such mouse is obtained using XenoMouse® technology (Abgenix; Fremont, Calif.), and is disclosed in U.S. Pat. Nos. 6,075,181, 6,091,001, and 6,114,598. For example, to produce the HCD122 antibody, mice transgenic for the human IgG1 heavy chain locus and the human κ light chain locus were immunized with Sf9 cells expressing human CD40. Mice can also be transgenic for other isotypes.

In some embodiments, the anti-CD40 antibody will have a light chain variable domain (V_{L}) that comprises the light chain CDR sequences of HCD122. Thus, in some embodiments, the anti-CD40 antibody will have a light chain variable domain that comprises an amino acid sequence as shown in SEQ ID NO:10 for CDR-L1, an amino acid sequence as shown in SEQ ID NO:11 for CDR-L2, and an amino acid sequence as shown in SEQ ID NO:12 for CDR-L3. In other embodiments, the anti-CD40 antibody will have a heavy chain variable domain (V_{H}) that comprises the heavy chain CDR sequences of HCD122. Thus, in some embodiments, the anti-CD40 antibody will have a heavy chain variable domain (V_{H}) that comprises an amino acid sequence as shown in SEQ ID NO:13 for CDR-H1, an amino acid sequence as shown in SEQ ID NO:14 for CDR-H2, and an amino acid sequence as shown in SEQ ID NO:15 for CDR-H3.

In further embodiments, the anti-CD40 antibody will have a light chain variable domain (V_{L}) that comprises the light chain CDR sequences of HCD122, and a heavy chain variable domain (V_{H}) that comprises the heavy chain CDR sequences of HCD122. Thus, in further embodiments, the anti-CD40 antibody will have a light chain variable domain (V_{L}) that comprises an amino acid sequence as shown in SEQ ID NO:10 for CDR-L1, an amino acid sequence as shown in SEQ ID NO:11 for CDR-L2, and an amino acid sequence as shown in SEQ ID NO:12 for CDR-L3, and a heavy chain variable domain (V_{H}) that comprises an amino acid sequence as shown in SEQ ID NO:13 for CDR-H1, an amino acid sequence as shown in SEQ ID NO:14 for CDR-H2, and an amino acid sequence as shown in SEQ ID NO:15 for CDR-H3.

There are various schemes for defining the CDR residues in a given antibody variable domain (e.g., see the web site designated as “bioinf.org.uk/abs” located on the World Wide Web (www)). The most commonly used is the Kabat numbering scheme (Kabat et al. (1991) Sequences of Proteins of Immunological Interest, 5th Ed. Public Health Service, National Institutes of Health, Bethesda, Md.). According to the Kabat numbering scheme, the CDRs in a light chain variable region are amino acids 24-34 (CDR-L1), 50-56 (CDR-L2) & 89-97 (CDR-L3), and the CDRs in a heavy chain variable region are amino acids 31-35 (CDR-H1), 50-65 (CDR-H2) and 95-102 (CDR-H3). Another well-known scheme is the Chothia numbering scheme (Chothia & Lesk (1987) MoI Biol. 196:901-917). By Chothia numbering, the CDRs in a light chain variable region are amino acids 26-32 (CDR-L1), 50-52 (CDR-L2) & 91-96 (CDR-L3), and the CDRs in a heavy chain variable region are amino acids 26-32 (CDR-H1), 53-55 (CDR-H2) and 96-101 (CDR-H3). Using one or more of the known schemes, the skilled person will readily be able to determine whether a given antibody meets the light chain and heavy chain CDR sequence requirements specified above.

“Antibody fragments” comprise a portion of an intact antibody, preferably the antigen-binding or variable region of the intact antibody. Examples of antibody fragments include Fab, F(ab′)₂, and Fv fragments.

By “Fab” is intended a monovalent antigen-binding fragment of an antibody that contains the constant domain of the light chain and the first constant domain (C_{H}1) of the heavy chain. Papain digestion of antibodies produces two identical Fab fragments, and a residual “Fc” fragment, whose name reflects its ability to crystallize readily. By “F(ab′)₂” is intended a bivalent antigen-binding fragment of an antibody that contains both light chains and part of both heavy chains, and which is retains the ability to cross-link antigen. Pepsin treatment yields an F(ab′)₂ fragment. “Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The invention may also use a single-chain Fv (scFv), which is a polypeptide comprising the V_{H} and V_{L} domains of an antibody, wherein these domains are present in a single polypeptide chain (see e.g., U.S. Pat. Nos. 4,946,778, 5,260,203, 5,455,030, and 5,856,456). Generally, the scFv polypeptide comprises a polypeptide linker between the V_{H} and V_{L} domains that enables the scFv to form the desired structure for antigen binding. For a review of scFv see Pluckthun (1994) in The Pharmacology of Monoclonal Antibodies, Vol. 113, ed. Rosenburg and Moore (Springer-Verlag, New York), pp. 269-315.

Fragments of an anti-CD40 antibody are suitable for use in the methods of the invention so long as they retain the ability to bind to the CD40 antigen on the surface of human B-cells. Such fragments are referred to herein as “antigen-binding” fragments. Such fragments are preferably characterized by functional properties similar to the corresponding full-length antibody. Thus, for example, a fragment of a full-length anti-CD40 antibody will preferably be capable of specifically binding a human CD40 antigen expressed on the surface of a human cell, and is free of significant agonist activity as described elsewhere herein. Fragments of an anti-CD40 antibody for use in the methods of the invention may in some instances retain the ability to bind to the relevant FcR or FcRs.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

The anti-CD40 antibodies used in the combination therapy of the invention are free of significant agonist activity when bound to CD40 antigen on the surface of human B-cells. In some embodiments, their binding to CD40 on the surface of human B-cells may result in inhibition of the proliferation and differentiation of the B-cells. The anti-CD40 antibodies suitable for use in the methods of the invention include those antibodies that can exhibit An “agonist” combines with a receptor on a cell and initiates a reaction or activity that is similar to or the same as that initiated by a natural ligand of the receptor. An agonist of CD40 induces any or all of, but not limited to, the following responses: B cell proliferation and/or differentiation; upregulation of intercellular adhesion via such molecules as ICAM-1, E-selectin, VCAM, and the like; secretion of pro-inflammatory cytokines such as IL-1, IL-6, IL-8, IL-12, TNF, and the like; signal transduction through the CD40 receptor by such pathways as TRAF (e.g., TRAF2 and/or TRAF3), MAP kinases such as NIK (NF-κB inducing kinase), 1-kappa B kinases (IKK α/β), transcription factor NF-κB, Ras and the MEK/ERK pathway, the PI3K/AKT pathway, the P38 MAPK pathway, and the like; transduction of an anti-apoptotic signal by such molecules as XIAP, mc1-1, bc1-x, and the like; B and/or T cell memory generation; B cell antibody production; B cell isotype switching, up-regulation of cell-surface expression of MHC Class II and CD80/86, and the like.

By “significant” agonist activity is intended an agonist activity of at least 30%, 35%, 40%, 45%, 50%, 60%, 70%, 75%, 80%, 85%, 90%, 95%, or 100% greater than the agonist activity induced by a negative control as measured in an assay of a B cell response. Preferably, “significant” agonist activity is an agonist activity that is at least 2-fold greater or at least 3-fold greater than the agonist activity induced by a negative control as measured in an assay of a B cell response. Thus, for example, where the B cell response of interest is B cell proliferation, “significant” agonist activity would be induction of a level of B cell proliferation that is at least 2-fold greater or at least 3-fold greater than the level of B cell proliferation induced by a negative control. In one embodiment, an antibody that does not bind to CD40 serves as the negative control. A substance “free of significant agonist activity” would exhibit an agonist activity of not more than about 25% greater than the agonist activity induced by a negative control, preferably not more than about 20% greater, 15% greater, 10% greater, 5% greater, 1% greater, 0.5% greater, or even not more than about 0.1% greater than the agonist activity induced by a negative control as measured in an assay of a B cell response.

An “antagonist” of CD40 prevents or reduces induction of any of the responses induced by binding of the CD40 receptor to an agonist ligand, particularly CD40L. The antagonist may reduce induction of a response to CD40L binding by 5%, 10%, 15%, 20%, 25%, 30%, 35%, preferably 40%, 45%, 50%, 55%, 60%, more preferably 70%, 80%, 85%, and most preferably 90%, 95%, 99%, or 100%.

Preferred antibodies and fragments for use in the methods of the invention are anti-CD40 antibodies that are free of significant agonist activity when bound to CD40 antigen on human B cells, and which exhibit antagonist activity when bound to CD40 antigen on human B cells. In some embodiments, the anti-CD40 antibody is free of significant agonist activity in one B cell response. In other embodiments, the anti-CD40 antibody is free of significant agonist activity in assays of more than one B cell response (e.g., proliferation and differentiation, or proliferation, differentiation, and antibody production).

Methods for measuring antagonist activity of an anti-CD40 therapeutic agent (e.g., an anti-CD40 antibody) are known in the art and include, but are not limited to, standard competitive binding assays, assays for monitoring antibody secretion by B cells, B cell proliferation assays, Banchereau-Like-B cell proliferation assays, T cell helper assays for antibody production, co-stimulation of B cell proliferation assays, and assays for up-regulation of B cell activation markers. Relevant assays are described in e.g., U.S. Pat. No. 6,087,329 and the international patent applications published as WO 00/75348, WO 2005/044294, WO 2005/044304, WO 2005/044305, WO 2005/044306, WO 2005/044307, WO 2005/044854, WO 2005/044855, WO 2006/073443, WO 2006/125117, WO 2006/125143, WO 2007/053661 and WO 2007/053767.

Any of the assays known in the art can be used to determine whether an anti-CD40 antibody acts as an antagonist of one or more B cell responses. In some embodiments, the anti-CD40 antibody acts as an antagonist of at least one B cell response selected from the group consisting of B cell proliferation, B cell differentiation, antibody production, intercellular adhesion, B cell memory generation, isotype switching, up-regulation of cell-surface expression of MHC Class II and CD80/86, and secretion of pro-inflammatory cytokines such as IL-8, IL-12, and TNF. Of particular interest are antagonist anti-CD40 antibodies that free of significant agonist activity with respect to B cell proliferation when bound to the human CD40 antigen on the surface of a human B cell.

The anti-CD40 antibody may be an antagonist of B cell proliferation induced by soluble or cell-surface CD40L, as measured in a B cell proliferation assay. Suitable B cell proliferation assays are known in the art. Suitable B cell proliferation assays are also described below. In some embodiments, the antagonist anti-CD40 antibody stimulates B cell proliferation at a level that is not more than about 25% greater than the B cell proliferation induced by a negative control (i.e., at least 75% inhibition), preferably not more than about 20% greater, 15% greater, 10% greater, 5% greater, 1% greater, 0.5% greater, or even not more than about 0.1% greater than the B cell proliferation induced by a negative control.

In other embodiments, the anti-CD40 antibody is an antagonist of B cell proliferation induced by another anti-CD40 antibody (e.g., the S2C6 anti-CD40 antibody; Kwekkeboom et al. (1993) Immunology 79:439-444), as measured in a B cell proliferation assay, and the level of B cell proliferation stimulated by the other anti-CD40 antibody in the presence of the antagonist anti-CD40 antibody is not more than about 25% of the B cell proliferation induced by the other anti-CD40 antibody in the absence of the antagonist anti-CD40 antibody (i.e., at least 75% inhibition), preferably not more than about 20%, 15%, 10%, 5%, 1%, 0.5%, or even not more than about 0.1% of the B cell proliferation induced by the other anti-CD40 antibody in the absence of the antagonist anti-CD40 antibody.

In yet other embodiments, the anti-CD40 antibody is an antagonist of B cell proliferation that is induced by the cell line EL4B5 (Kwekkeboom et al. (1993) Immunology 79:439-444) as measured in a B cell activation assay, and the level of B cell proliferation stimulated by the EL4B5 cell line in the presence of the antagonist anti-CD40 antibody is not more than about 25% of the B cell proliferation induced by this cell line in the absence of the antagonist anti-CD40 antibody (i.e., at least 75% inhibition), preferably not more than about 20%, 15%, 10%, 5%, 1%, 0.5%, or even not more than about 0.1% of the B cell proliferation induced by this cell line in the absence of the antagonist anti-CD40 antibody.

In still other embodiments, the anti-CD40 antibody is an antagonist of human T-cell-induced antibody production by human B cells as measured in the human T-cell helper assay for antibody production by B cells. In this manner, the level of IgG antibody production, IgM antibody production, or both IgG and IgM antibody production by B cells stimulated by T cells in the presence of the antagonist anti-CD40 antibody is not more than about 50% of the respective antibody production by B cells stimulated by T cells in the absence of the antagonist anti-CD40 antibody (i.e., at least 75% inhibition), preferably not more than about 25%, 20%, 15%, 10%, 5%, 1%, 0.5%, or even not more than about 0.1% of the respective antibody production by B cells stimulated by T cells in the absence of the antagonist anti-CD40 antibody.

For example, the following assays can be used to assess the antagonist activity of an anti-CD40 antibody. Human B cells for these assays can be obtained, for example, by isolation from tonsils obtained from individuals undergoing tonsillectomies, essentially as described in De Groot et al. (1990) Lymphokine Research (1990) 9:321. Briefly, the tissue is dispersed with scalpel blades, phagocytic and NK cells are depleted by treatment with 5 mM L-leucine methyl ester and T cells are removed by one cycle of rosetting with sheep erythrocytes (SRBC) treated with 2-aminoethyl isothiouronium bromide. The purity of the resulting B lymphocyte preparations can be checked by indirect immunofluorescent labelling with anti-(CD20) mAb B1 (Coulter Clone, Hialeah, FA) or anti-(CD3) mAb OKT3 (Ortho, Raritan, N.J.) and a FITC-conjugated F(ab′)₂ fragment of rabbit anti-(mouse Ig) (Zymed, San Francisco, Calif.), and FACS analysis.

### B-cell Proliferation Assay

B cells (4×10⁴ per well) are cultured in 200 μl IMDM supplemented with 10% fetal calf serum in flat bottom 96-well microtiter plates. B cells are stimulated by addition of immobilized anti-(IgM) antibodies (Immunobeads; 5 μg/ml; BioRad, Richmond, Calif.). Where desired, 100 U/ml recombinant IL-2 is added. Varying concentrations of test monoclonal antibodies (mAbs) are added at the onset of the microcultures and proliferation is assessed at day 3 by measurement of the incorporation of (3H)-thymidine after 18 hour pulsing. An antagonist anti-CD40 antibody does not significantly costimulate human B-cell proliferation in the presence of immobilized anti-IgM or in the presence of immobilized anti-IgM and IL-2.

### Banchereau-Like B-Cell Proliferation Assay

For testing the ability of anti-CD40 monoclonal antibodies to stimulate B-cell proliferation in a culture system analogous to that described by Banchereau et al. (1991) Science (1991) 251:70, mouse 3T6 transfectant cells expressing the HR allellic form of human FcγRI are used. B cells (2×10⁴ per well) are cultured in flat-bottom microwells in the presence of 1×10⁴ transfectant cells (irradiated with 5000 Rad) in 200 μl IMDM supplemented with 10% fetal calf serum and 100 U/ml recombinant IL-4. Before addition of the B cells, the 3T6 cells are allowed to adhere to the culture plastic for at least 5 hours. Anti-CD40 mAbs are added at concentrations varying from 15 ng/ml to 2000 ng/ml and proliferation of B cells is assessed by measurement of thymidine incorporation at day 7, upon 18 hour pulsing with [³H]thymidine.

Inhibition of S2C6-Stimulated B-Cell Proliferation Using Antagonist Anti-CD40 mAbs

Antagonist anti-CD40 monoclonal antibodies (mAbs) may also be characterized by their ability to inhibit stimulation of B-cell proliferation by an anti-CD40 antibody such as S2C6 (also known as SGN-14, which is reportedly an agonist of CD40 stimulation of proliferation of normal B cells; Francisco et al. (2000) Cancer Res. 60:3225-3231) using the B-cell Proliferation Assay described above. Human tonsillar B cells (4×10⁴ per well) are cultured in 200 μl in microwells in the presence of anti-IgM coupled to Sepharose beads (5 μg/ml) and anti-CD40 mAb S2C6 (1.25 μg/ml). Varying concentrations of an anti-CD40 mAb of interest are added and [³H]-thymidine incorporation is assessed after 3 days. As a control anti-(glucocerebrosidase) mAb 8E4 can be added in similar concentrations. Barneveld et al. (1983) Eur. J. Biochem. 134:585. An antagonist anti-CD40 antibody can inhibit the costimulation of anti-IgM induced human B-cell proliferation by mAb S2C6, for example, by at least 75% or more (i.e., S2C6-stimulated proliferation in the presence of an antagonist anti-CD40 antibody is no more than 25% of that observed in the absence of the antagonist anti-CD40 antibody). In contrast, no significant inhibition would be seen with equivalent amounts of non-relevant mAb 8E4, directed to β-glucocerebrosidase. Barneveld et al., supra. Such a result would indicate that the anti-CD40 mAbs does not deliver stimulatory signals for the proliferation of human B cells, but, conversely, can inhibit stimulatory signals exerted by triggering CD40 with another mAb.

B-Cell Activation Assay with EL4B5 Cells

Zubler et al. (1985) J. Immunol. (1985) 134:3662 observed that a mutant subclone of the mouse thymoma EL-4 line, known as EL4B5, could strongly stimulate B cells of both murine and human origin to proliferate and differentiate into immunoglobulin-secreting plasma cells in vitro. This activation was found to be antigen-independent and not MHC restricted. For optimal stimulation of human B cells, the presence of supernatant from activated human T cells was needed but a B-cell response also occurred when EL4B5 cells were preactivated with phorbol-12-myristate 13-acetate (PMA) or IL-1. Zubler et al. (1987) Immunological Reviews 99:281; and Zhang et al. (1990) J. Immunol. 144:2955. B-cell activation in this culture system is efficient—limiting dilution experiments have shown that the majority of human B cells can be activated to proliferate and differentiate into antibody-secreting cells. Wen et al. (1987) Eur. J. Immunol. 17:887.

B cells (1000 per well) are cultured together with irradiated (5000 Rad) EL4B5 cells (5×10⁴ per well) in flat bottom microtiter plates in 200 μl IMDM supplemented with 10% heat-inactivated fetal calf serum, 5 ng/ml phorbol-12-myristate 13-acetate and 5% human T-cell supernatant. mAbs are added at varying concentrations at the onset of the cultures and thymidine incorporation is assessed at day 6 after 18 hour pulsing with [³H]-thymidine. For the preparation of T-cell supernatant, purified T cells are cultured at a density of 10⁶/ml for 36 hours in the presence of 1 μg/ml PHA and 10 ng/ml PMA. Wen et al. (1987) Eur. J. Immunol. (1987) 17:887. T-cell supernatant is obtained by centrifugation of the cells and stored at −20° C. The effectiveness of T-cell supernatants in enhancing proliferation of human B cells in EL4B5-B cell cultures is tested and the most effective supernatants are pooled for use in experiments. When assessing the effect of an anti-CD40 antibody on EL4B5-induced human B-cell proliferation, a monoclonal antibody such as MOPC-141 (IgG2b) can be added as a control.

### Human T Cell Helper Assay for Antibody Production by B Cells

An antagonist anti-CD40 antibody may function as an antagonist of antibody production by B cells. An anti-CD40 antibody can be tested for this type of antagonist activity by assessing the antibody's ability to inhibit antibody production by B cells that have been stimulated in a contact-dependent manner with activated T cells in a T cell helper assay. In this manner, 96-well tissue culture plates are coated with a 1:500 dilution of ascites fluid of anti-CD3 mAb CLB-T3/3 (CLB, Amsterdam, The Netherlands). As indicated costimulatory mAbs are added: anti CD2 mAbs CLB-T11.1/1 and CLB-T11.2/1 (CLB, Amsterdam, The Netherlands), both ascites 1:1000 and anti-CD28 mAb CLB-28/1 (CLB, Amsterdam, The Netherlands). Subsequently, tonsillar T cells (irradiated, 3000 Rad; 10⁵ per well), tonsillar B cells (10⁴ per well), and rIL-2 (20 U/ml) are added. The final volume of each cell culture is 200 μl. After 8 days, cells are spun down, and cell-free supernatant is harvested. The concentrations of human IgM and IgG in (diluted) samples is estimated by ELISA as described below.

In one embodiment, human tonsillar B cells (10⁴/well) are cultured together with irradiated purified T cells (3000 rad, 10⁵/well) in 96-well plates, coated with anti-CD3 mAb and with or without different mAbs to costimulate the T cells. After 8 days of culture the supernatants are harvested for the determination of antibody production by the B cells. Antibody production by the B cells is assessed by the ELISA assay described below. The anti-CD40 antibody of interest is added in varying concentrations from the onset of the cultures. As a control, mAb MOPC-141 can be added.

An antagonist anti-CD40 antibody can inhibit IgG and IgM antibody production of B cells stimulated by human T cells by at least 50% or more (i.e., T cell-induced antibody production by B cells in the presence of an antagonist anti-CD40 antibody is no more than 50% of that observed in the absence of the antagonist anti-CD40 antibody). In contrast, a control antibody such as MOPC-141 would have no significant effect on T cell-induced antibody production by B cells.

### ELISA Assay for Antibody Quantification

The concentrations of human IgM and IgG are estimated by ELISA. 96-well ELISA plates are coated with 4 μg/ml mouse anti-human IgG mAb MH 16-01 (CLB, Amsterdam, The Netherlands) or with 1.2 μg/ml mouse anti-human IgM mAb 4102 (Tago, Burlingame, Calif.) in 0.05 M carbonate buffer (pH=9.6), by incubation for 16 h at 4° C. Plates are washed 3 times with PBS-0.05% Tween-20 (PBS-Tween) and saturated with BSA for 1 hour. After 2 washes the plates are incubated for 1 h at 37° C. with different dilutions of the test samples. After 3 washes, bound Ig is detected by incubation for 1 h at 37° C. with 1 μg/ml peroxidase-labeled mouse anti-human IgG mAb MH 16-01 (CLB) or mouse anti-human IgM mAb MH 15-01 (CLB). Plates are washed 4 times and bound peroxidase activity is revealed by the addition of O-phenylenediamine as a substrate. Human standard serum (H00, CLB) is used to establish a standard curve for each assay.

Antagonist anti-CD40 antibodies are known in the art. See, for example, the human anti-CD40 antibody produced by the hybridoma designated F4-465 disclosed in U.S. Patent Application Publication Nos. 20020142358 and 20030059427. F4-465 was obtained from the HAC mouse (Kuroiwa et al. (2000) Nature Biotech. 10:1086 (2000)) and therefore expresses the human lambda light chain.

In addition to antagonist activity, the anti-CD40 antibody for use in the methods of the present invention will preferably have another mechanism of action against a target cell. The anti-CD40 antibody will preferably have ADCC activity.

Of particular interest to the present invention are anti-CD40 antibodies that share the binding characteristics of HCD122 (produced by the hybridoma cell line deposited with the ATCC (American Type Culture Collection; 10801 University Blvd., Manassas, Va. 20110-2209 (USA)) on Sep. 17, 2003, as Patent Deposit No. PTA-5543). Such antibodies include, but are not limited to:

a) the monoclonal antibody HCD122, produced by the hybridoma cell line deposited with the ATCC as Patent Deposit No. PTA-5543;

b) an antibody comprising an amino acid sequence selected from the group consisting of the sequence shown in SEQ ID NO:2, the sequence shown in SEQ ID NO:4, the sequence shown in SEQ ID NO:5, both the sequences shown in SEQ ID NO:2 and SEQ ID NO:4, and both the sequences shown in SEQ ID NO:2 and SEQ ID NO:5;

c) an antibody comprising an amino acid sequence selected from the group consisting of the sequence shown in SEQ ID NO:17, the sequence shown in SEQ ID NO:19, the sequence shown in SEQ ID NO:20, both the sequences shown in SEQ ID NO:17 and SEQ ID NO:19, and both the sequences shown in SEQ ID NO:17 and SEQ ID NO:20;

d) an antibody comprising an amino acid sequence selected from the group consisting of the sequence shown in SEQ ID NO:16, the sequence shown in SEQ ID NO:18, and both the sequences shown in SEQ ID NO:16 and SEQ ID NO:18;

e) an antibody having an amino acid sequence encoded by a nucleic acid molecule comprising a nucleotide sequence selected from the group consisting of the sequence shown in SEQ ID NO:1, the sequence shown in SEQ ID NO:3, and both the sequences shown in SEQ ID NO:1 and SEQ ID NO:3;

f) an antibody having a light chain variable domain (V_{L}) that comprises the amino acid sequence as shown in SEQ ID NO:10 for CDR-L1, the amino acid sequence as shown in SEQ ID NO:11 for CDR-L2, and the amino acid sequence as shown in SEQ ID NO:12 for CDR-L3;

g) an antibody having a heavy chain variable domain (V_{H}) that comprises the amino acid sequence as shown in SEQ ID NO:13 for CDR-H1, the amino acid sequence as shown in SEQ ID NO:14 for CDR-H2, and the amino acid sequence as shown in SEQ ID NO:15 for CDR-H3;

h) an antibody having a light chain variable domain (V_{L}) that comprises the amino acid sequence as shown in SEQ ID NO:10 for CDR-L1, the amino acid sequence as shown in SEQ ID NO:11 for CDR-L2, and the amino acid sequence as shown in SEQ ID NO:12 for CDR-L3, and having a heavy chain variable domain (V_{H}) that comprises the amino acid sequence as shown in SEQ ID NO:13 for CDR-H1, the amino acid sequence as shown in SEQ ID NO:14 for CDR-H2, and the amino acid sequence as shown in SEQ ID NO:15 for CDR-H3;

i) an antibody that binds domain 2 of human CD40 antigen;

j) an antibody that binds to a CD40 epitope capable of binding the monoclonal antibody HCD122;

k) an antibody that binds to an epitope comprising residues 82-87 of the human CD40 sequence shown in SEQ ID NO:7 or SEQ ID NO:9; and

l) an antibody that competes with the monoclonal antibody HCD122 in a competitive binding assay.

An anti-CD40 antibody obtained from a CHO cell containing one or more expression vectors encoding the antibody can be used in the methods of the invention.

The monoclonal antibody HCD122, produced by the hybridoma cell line deposited with the ATCC as Patent Deposit No. PTA-5543, is particularly preferred for use in the methods of the invention.

The monoclonal antibody HCD122 binds domain 2 of human CD40 antigen, whereas earlier anti-CD40 antibodies having antagonistic properties were found to bind to other domains of human CD40.

The HCD122 monoclonal antibody binds soluble CD40 in ELISA-type assays, prevents the binding of CD40-ligand to cell-surface CD40, and displaces the pre-bound CD40-ligand, as determined by flow cytometric assays. When tested in vitro for effects on proliferation of B cells from normal human subjects, HCD122 acts as antagonist anti-CD40 antibody. Furthermore, HCD122 does not induce strong proliferation of human lymphocytes from normal subjects. The antibody is able to kill CD40-expressing target cells by antibody dependent cellular cytotoxicity (ADCC). The binding affinity of HCD122 for human CD40 is 5×10⁻¹⁰ M, as determined by the Biacore™ assay.

The nucleotide and amino acid sequences of the HCD122 antibody are known (e.g., see WO 2005/044854). Further, the mouse hybridoma line 153.8E2.D10.D6.12.12 (CMCC#12056), which expresses the HCD122 antibody, has been deposited with the American Type Culture Collection [ATCC; 10801 University Blvd., Manassas, Va. 20110-2209 (USA)] on Sep. 17, 2003, under Patent Deposit Number PTA-5543.

The complete sequence for the light chain of HCD122 is set forth in SEQ ID NO:2, which includes the leader sequence (residues 1-20 of SEQ ID NO:2), the variable region (residues 21-132 of SEQ ID NO:2), and the constant region (residues 133-239 of SEQ ID NO:2). The complete sequence for the heavy chain of HCD122 is set forth in SEQ ID NO:4, which includes the leader sequence (residues 1-19 of SEQ ID NO:4), the variable region (residues 20-139 of SEQ ID NO:4), and the constant regions (residues 140-469 of SEQ ID NO:4). The complete sequence for a variant of HCD122 is set forth in SEQ ID NO:5, which includes the leader sequence (residues 1-19 of SEQ ID NO:5), the variable region (residues 20-139 of SEQ ID NO:5), and the constant regions (residues 140-469 of SEQ ID NO:5). This variant differs from HCD122 in that it contains a substitution of a serine residue for the alanine residue at position 153 of SEQ ID NO:4, which is within the constant regions. The nucleotide sequences encoding the light and heavy chains of HCD122 are set forth in SEQ ID NO:1 (coding sequence for the light chain of HCD122) and SEQ ID NO:3 (coding sequence for the heavy chain of HCD122).

The amino acid sequence for the variable region of the HCD122 light chain without the leader sequence (i.e., residues 21-132 of SEQ ID NO:2) is set forth in SEQ ID NO:16. The amino acid sequence for the variable and constant regions of the HCD122 light chain without the leader sequence (i.e., residues 21-239 of SEQ ID NO:2) is set forth in SEQ ID NO:17. The amino acid sequence for the variable region of the HCD122 heavy chain without the leader sequence (i.e., residues 20-139 of SEQ ID NO:4) is set forth in SEQ ID NO:18. The amino acid sequence for the variable and constant regions of the HCD122 heavy chain without the leader sequence (i.e., residues 20-469 of SEQ ID NO:4) is set forth in SEQ ID NO:19. The amino acid sequence for the variable and constant regions of the HCD122 heavy chain variant (i.e., residues 20-469 of SEQ ID NO:5) is set forth in SEQ ID NO:20.

Anti-CD40 antibodies for use in the methods of the present invention include antibodies differing from the HCD122 monoclonal antibody but retaining the CDRs, and antibodies with one or more amino acid addition(s), deletion(s), or substitution(s). HCD122 is a fully human antibody, but can be further de-immunized if desired. De-immunized anti-CD40 antibodies can be produced using known methods, e.g., as described in WO 98/52976 and WO 00/34317. In this manner, residues within the anti-CD40 antibodies may be modified so as to render the antibodies less immunogenic to humans while retaining their therapeutic activity.

Any known antibody having the binding specificity of interest can have sequence variations produced using methods described in, for example, EP 0983303, WO 00/34317, and WO 98/52976. For example, it has been shown that sequences within the CDR can cause an antibody to bind to MHC Class II and trigger an unwanted helper T-cell response in certain patients. A conservative substitution can allow the antibody to retain binding activity yet lose its ability to trigger an unwanted T-cell response. Any such conservative or non-conservative substitutions can be made using art-recognized methods, such as those noted elsewhere herein, and the resulting antibodies can also be used in the methods of the present invention. The variant antibodies can be routinely tested for the particular activity, for example, antagonist activity, affinity, and specificity using methods described herein.

For example, amino acid sequence variants of an antagonist anti-CD40 antibody, for example, the HCD122 monoclonal antibody, can be prepared by mutations in the cloned DNA sequence encoding the antibody of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, N.Y.); U.S. Pat. No. 4,873,192; and the references cited therein. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.). Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of an antibody of interest, for example, an antagonist anti-CD40 antibody polypeptide of interest, modifications may be made such that variants continue to possess the desired activity, i.e., similar binding affinity and, in the case of antagonist anti-CD40 antibodies, are capable of specifically binding to a human CD40 antigen expressed on the surface of a human cell, and being free of significant agonist activity but exhibiting antagonist activity when bound to a CD40 antigen on a human CD40-expressing cell. Obviously, any mutations made in the DNA encoding the variant polypeptide must not place the sequence out of reading frame and preferably will not create complementary regions that could produce secondary mRNA structure (e.g., see EP 0075444).

In addition, the constant region of an antibody, for example, an antagonist anti-CD40 antibody, can be mutated to alter effector function in a number of ways. For example, see U.S. Pat. No. 6,737,056 B1 and U.S. Patent Application Publication No. 2004/0132101A1, which disclose Fc mutations that optimize antibody binding to Fc receptors.

Preferably, variants of a reference antibody, for example, an antagonist anti-CD40 antibody, have amino acid sequences that have at least 70% or 75% sequence identity, preferably at least 80% or 85% sequence identity, more preferably at least 90%, 91%, 92%, 93%, 94% or 95% sequence identity to the amino acid sequence for the reference antibody, for example, an antagonist anti-CD40 antibody molecule, for example, the HCD122 monoclonal antibody described herein. More preferably, the molecules share at least 96%, 97%, 98% or 99% sequence identity. For purposes of the present invention, percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ from the reference antibody, for example, an antagonist anti-CD40 antibody, by as few as 1 to 15 amino acid residues, as few as 1 to 10 amino acid residues, such as 6-10, as few as 5, as few as 4, 3, 2, or even 1 amino acid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm).

The precise chemical structure of an antibody capable of specifically binding CD40 and retaining antagonist activity, particularly when bound to CD40 antigen on malignant B cells, depends on a number of factors. As ionizable amino and carboxyl groups are present in an antibody molecule, a particular polypeptide may be obtained as an acidic or basic salt, or in neutral form. All such preparations that retain their biological activity when placed in suitable environmental conditions are included in the definition of antagonist anti-CD40 antibodies as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an anti-CD40 antibody used herein. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity.

The art provides substantial guidance regarding the preparation and use of antibody variants. In preparing the anti-CD40 antibody variants, one of skill in the art can readily determine which modifications to the native protein nucleotide or amino acid sequence will result in a variant that is suitable for use as a therapeutically active component of a pharmaceutical composition used in the methods of the present invention.

The anti-CD40 antibody for use in the methods of the invention preferably possesses at least one of the following biological activities in vitro and/or in vivo: inhibition of antibody secretion by normal human peripheral B cells stimulated by T cells; inhibition of survival and/or proliferation of normal human peripheral B cells stimulated by CD40L-expressing cells or soluble CD40 ligand (sCD40L); inhibition of survival and/or proliferation of normal human peripheral B cells stimulated by Jurkat T cells; inhibition of “survival” anti-apoptotic intracellular signals in any cell stimulated by sCD40L or solid-phase CD40L; and, inhibition of CD40 signal transduction in any cell upon ligation with sCD40L or solid-phase CD40L, deletion, anergy and/or tolerance induction of CD40-bearing target cells or cells bearing cognate ligands to CD40 including, but not limited to, T cells and B cells, induction of expansion or activation of CD4⁺CD25⁺ regulatory T cells (see for example, donor alloantigen-specific tissue rejection via CD40-CD40L interference, van Maurik et al. (2002) J. Immunol. 169:5401-5404), cytotoxicity via any mechanism (including, but not limited to, antibody-dependent cell-mediated cytotoxicity (ADCC), complement-dependent cytotoxicity (CDC), down-regulation of proliferation, and/or apoptosis in target cells), modulation of target cell cytokine secretion and/or cell surface molecule expression, and combinations thereof. Assays for such biological activities can be performed as described herein. See also the assays described in Schultze et al. (1998) Proc. Natl. Acad. Sci. USA 92:8200-8204; Denton et al. (1998) Pediatr. Transplant. 2:6-15; Evans et al. (2000) J. Immunol. 164:688-697; Noelle (1998) Agents Actions Suppl. 49:17-22; Lederman et al. (1996) Curr. Opin. Hematol. 3:77-86; Coligan et al. (1991) Current Protocols in Immunology 13:12; Kwekkeboom et al. (1993) Immunology 79:439-444; and U.S. Pat. Nos. 5,674,492 and 5,847,082.

It is possible to engineer an antibody to have increased ADCC activity. In particular, the carboxy-terminal half of the CH2 domain is important for ADCC mediated through the FcRIII receptor. Since the CH2 and hinge regions have an important role in effector functions, a series of multiple-domain antibodies that contain extra CH2 and/or hinge regions may be created and investigated for any changes in effector potency (see Greenwood et al. (1994) Ther. Immunol. 1(5):247-55). An alternative approach may be to engineer extra domains in parallel, for example, through creation of dimers by engineering a cysteine into the H-chain of a chimeric Ig (see Shopes (1992) J. Immunol. 148(9):2918-2922). Furthermore, changes to increase ADCC activity may be engineered by introducing mutations into the Fc region (see, for example, U.S. Pat. No. 6,737,056 B1), expressing cells in fucosyl transferase deficient cell lines (see, for example, U.S. Patent Application Publication No. 2003/0115614), or effecting other changes to antibody glycosylation (see, for example, U.S. Pat. No. 6,602,684).

A representative assay to detect antagonist anti-CD40 antibodies specific to the CD40-antigen epitopes identified herein is a “competitive binding assay”. Competitive binding assays are serological assays in which unknowns are detected and quantitated by their ability to inhibit the binding of a labeled known ligand to its specific antibody. This is also referred to as a competitive inhibition assay. In a representative competitive binding assay, labeled CD40 polypeptide is precipitated by candidate antibodies in a sample, for example, in combination with monoclonal antibodies raised against one or more epitopes of anti-CD40 monoclonal antibodies. Anti-CD40 antibodies that specifically react with an epitope of interest can be identified by screening a series of antibodies prepared against a CD40 protein or fragment of the protein comprising the particular epitope of the CD40 protein of interest. For example, for human CD40, epitopes of interest include epitopes comprising linear and/or nonlinear amino acid residues of the short isoform of human CD40 (see GenBank Accession No. NP_{—}690593) set forth in SEQ ID NO:7, encoded by the sequence set forth SEQ ID NO:6; see also GenBank Accession No. NM_{—}152854), or of the long isoform of human CD40 (see GenBank Accession Nos. CAA43045 and NP_{—}001241, set forth in SEQ ID NO:9, encoded by the sequence set forth in SEQ ID NO:8; see GenBank Accession Nos. X60592 and NM_{—}001250). Alternatively, competitive binding assays with previously identified suitable antagonist anti-CD40 antibodies could be used to select monoclonal antibodies comparable to the previously identified antibodies.

Antibodies employed in such immunoassays may be labeled or unlabeled. Unlabeled antibodies may be employed in agglutination; labeled antibodies may be employed in a wide variety of assays, employing a wide variety of labels. Detection of the formation of an antibody-antigen complex between an anti-CD40 antibody and an epitope of interest can be facilitated by attaching a detectable substance to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or ³H. Such labeled reagents may be used in a variety of well-known assays, such as radioimmunoassays, enzyme immunoassays, e.g., ELISA, fluorescent immunoassays, and the like. See for example, U.S. Pat. Nos. 3,766,162; 3,791,932; 3,817,837; and 4,233,402.

As noted above, the combination therapy of the invention addresses problems associated with known therapies for diseases or conditions associated with neoplastic B-cell growth, including therapy using rituximab (commercially available under the tradename Rituxan®). Rituximab has been shown to be an effective treatment for low-, intermediate-, and high-grade non-Hodgkin's lymphoma (NHL) and active in other B-cell malignancies (see for example, Maloney et al. (1994) Blood 84:2457-2466), McLaughlin et al. (1998) J. Clin. Oncol. 16:2825-2833, Maloney et al. (1997) Blood 90:2188-2195, Hainsworth et al. (2000) Blood 95:3052-3056, Colombat et al. (2001) Blood 97:101-106, Coiffer et al. (1998) Blood 92:1927-1932), Foran et al. (2000) J. Clin. Oncol. 18:317-324, Anderson et al. (1997) Biochem. Soc. Trans. 25:705-708, or Vose et al. (1999) Ann. Oncol. 10:58a). Rituximab is licensed for treatment of relapsed B cell low-grade or follicular non-Hodgkin's lymphoma (NHL). Some patients become resistant to treatment with rituximab (see Witzig et al. (2002) J. Clin. Oncol. 20:3262, Grillo-Lopez et al. (1998) J. Clin. Oncol. 16:2825, or Jazirehi et al. (2003) Mol. Cancer. Ther 2:1183-1193). For example, some patients lose CD20 expression on malignant B cells after anti-CD20 antibody therapy (Davis et al. (1999) Clin. Cancer Res. 5:611). Furthermore, 30% to 50% of patients with low-grade NHL exhibit no clinical response to this monoclonal antibody (Hainsworth et al. (2000) Blood 95:3052-3056; Colombat et al. (2001) Blood 97:101-106). For patients developing resistance to this monoclonal antibody, or having a B-cell lymphoma that is resistant to initial therapy with this antibody, alternative forms of therapeutic intervention are needed. Alternative therapies are also desirable for patients who relapse after therapy with rituximab. The discovery of antibodies with superior therapeutic, in particular anti-tumor, activity compared to rituximab could drastically improve methods of therapy for diseases or condition associated with neoplastic B cell growth, such as B cell lymphomas, particularly B cell non-Hodgkin's lymphoma.

In some embodiments, the combination therapy of the invention provides a more potent therapeutic effect than rituximab, e.g., where anti-tumor activity is assayed with equivalent amounts of these antibodies in a nude mouse xenograft tumor model using human lymphoma or myeloma cell lines. In other embodiments, the combination therapy of the invention provides a more potent therapeutic effect than combination therapy with rituximab and CHOP (commonly known as R-CHOP), e.g., where anti-tumor activity is assayed with equivalent amounts of these antibodies in a nude mouse xenograft tumor model using human lymphoma or myeloma cell lines.

Suitable nude mouse xenograft tumor models include those using the human Burkitt's lymphoma cell lines known as Namalwa and Daudi. Preferred embodiments assay anti-tumor activity in a staged nude mouse xenograft tumor model using the Daudi human lymphoma cell line. A staged nude mouse xenograft tumor model using the Daudi lymphoma cell line is more effective at distinguishing the therapeutic efficacy of a given antibody than is an unstaged model, as in the staged model antibody dosing is initiated only after the tumor has reached a measurable size. In the unstaged model, antibody dosing is initiated generally within about 1 day of tumor inoculation and before a palpable tumor is present. The ability of an antibody to outperform rituximab or R-CHOP (i.e., to exhibit increased therapeutic activity) in a staged model is a strong indication that the antibody will be more therapeutically effective than rituximab. Moreover, in the Daudi model, anti-CD20, the target for rituximab is expressed on the cell surface at a higher level than is CD40.

In the examples herein, the inventors used the RL (ATCC; CRL-2261) and SU-DHL-4 (DSMZ; ACC 495) human B-cell lymphoma cell lines. These cells lines are both reported to be negative for the Epstein-Barr virus genome, in contrast to many of the common lymphoma cell lines used in the field. The use of cell lines that are positive for the Epstein-Barr virus may lead to problems when interpreting experimental data, due to influences on signalling by the oncogenic EBV in those cell lines. The RL and SU-DHL-4 lymphoma cell lines were specifically chosen by the inventors because they are EBV negative, which allows greater confidence that the results are indeed authentic, i.e., predictive of therapeutic efficacy in humans.

Accordingly, in some embodiments, the combination therapy of the invention provides a more potent therapeutic effect than rituximab, where anti-tumor activity is assayed with equivalent amounts of the antibodies in a nude mouse xenograft tumor model using a human lymphoma cell line that is negative for the Epstein-Barr virus genome. In further embodiments, the combination therapy of the invention provides a more potent therapeutic effect than combination therapy with rituximab and CHOP, where anti-tumor activity is assayed with equivalent amounts of the antibodies in a nude mouse xenograft tumor model using a human lymphoma cell line that is negative for the Epstein-Barr virus genome. In these embodiments, the RL or SU-DHL-4 lymphoma cell lines may be used.

By “equivalent amount” of an anti-CD40 antibody and rituximab is intended the same mg dose is administered on a per weight or per volume basis. Thus, where the anti-CD40 antibody is dosed at 0.01 mg/kg body weight of the mouse used in the tumor model, rituximab is also dosed at 0.01 mg/kg body weight of the mouse.

Another difference in antibody efficacy is to measure in vitro the concentration of antibody needed to obtain the maximum lysis of tumor cells in vitro in the presence of NK cells. For example, the anti-CD40 antibodies may reach maximum lysis of Daudi cells at an EC50 of less than ½, and preferably ¼, and most preferably, 1/10 the concentration of rituximab. The anti-CD40 antibody or antigen-binding fragment thereof may therefore be more potent than an equivalent amount of rituximab in an assay of antibody-dependent cellular cytotoxicity (ADCC), e.g., an assay that comprises incubating CD40-expressing cells and CD20-expressing cells with isolated human natural killer (NK) cells in the presence of the relevant antibody, as described in WO 2007/053767.

The invention uses anti-CD40 antibodies for treating diseases or conditions associated with neoplastic B-cell growth.

The anti-CD40 antibodies of this invention are administered at a concentration that is therapeutically effective to treat a disease or condition associated with neoplastic CD40 expressing B cells. To accomplish this goal, the antibodies may be formulated using a variety of acceptable carrier and/or excipients known in the art. The anti-CD40 antibody may be administered by a parenteral route of administration. Typically, the antibodies are administered by injection, either intravenously or subcutaneously. Methods to accomplish this administration are known to those of ordinary skill in the art.

Intravenous administration occurs preferably by infusion over a period of about less than 1 hour to about 10 hours (more preferably less than 1, 2, 3, 4, 5, 6, 7, 8, 9, or 10 hours). Subsequent infusions may be administered over a period of about less than 1 to about 6 hours, including, for example, about 1 to about 4 hours, about 1 to about 3 hours, or about 1 to about 2 hours. Alternatively, a dose can be administered subcutaneously.

A pharmaceutical composition of the invention is formulated to be compatible with its intended route of administration. Solutions or suspensions used for parenteral application can include the following components: a sterile diluent such as water for injection, saline solution; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

The anti-CD40 antibodies are typically provided by standard technique within a pharmaceutically acceptable buffer, for example, sterile saline, sterile buffered water, combinations of the foregoing, etc. Methods for preparing parenterally administrable agents are described in Remington: The Science and Practice of Pharmacy (21st edition, Lippincott Williams & Wilkins, May 2005). See also, for example, WO 98/56418, which describes stabilized antibody pharmaceutical formulations suitable for use in the methods of the present invention.

The amount of at least one anti-CD40 antibody to be administered is readily determined by one of ordinary skill in the art. Factors influencing the mode of administration and the respective amount of at least one anti-CD40 antibody include, but are not limited to, the severity of the disease, the history of the disease, and the age, height, weight, health, type of disease, and physical condition of the individual undergoing therapy or response to antibody infusion. Similarly, the amount of anti-CD40 antibody to be administered will be dependent upon the mode of administration and whether the subject will undergo a single dose or multiple doses of this anti-tumor agent. Generally, a higher dosage of anti-CD40 antibody is preferred with increasing weight of the subject undergoing therapy.

For a single dose of anti-CD40 antibody to be administered may be in the range from about 0.1 mg/kg to about 35 mg/kg, from about 0.5 mg/kg to about 30 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, or from about 5 mg/kg to about 15 mg/kg. Thus, for example, the dose can be 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, or 35 mg/kg, or other such doses falling within the range of about 0.3 mg/kg to about 35 mg/kg.

Treatment of a subject with a therapeutically effective amount of an antibody can include a single treatment or, preferably, can include a series of treatments. Thus, the methods of the invention may comprise administration of multiple doses of anti-CD40 antibody. The method may comprise administration of 1, 2, 3, 4, 5, 6, 7, 8, 9, 10, 15, 20, 25, 30, 35, 40, or more therapeutically effective separate doses of a pharmaceutical composition comprising an anti-CD40 antibody. The frequency and duration of administration of multiple doses of the pharmaceutical compositions comprising anti-CD40 antibody can be readily determined by one of skill in the art without undue experimentation. The same therapeutically effective dose of an anti-CD40 antibody can be administered over the course of a treatment period. Alternatively, different therapeutically effective doses of an anti-CD40 antibody can be used over the course of a treatment period.

In an example, a subject is treated with anti-CD40 antibody in the range of between about 0.1 to 20 mg/kg body weight, once per week for between about 1 to 10 weeks, preferably between about 2 to 8 weeks, more preferably between about 3 to 7 weeks, and even more preferably for about 4, 5, or 6 weeks. Treatment may occur at intervals of every 2 to 12 months to prevent relapse or upon indication of relapse. It will also be appreciated that the effective dosage of antibody used for treatment may increase or decrease over the course of a particular treatment. Changes in dosage may result and become apparent from the results of diagnostic assays.

Thus, in one embodiment, the dosing regimen includes a first administration of a therapeutically effective dose of at least one anti-CD40 antibody on days 1, 8, 15, and 22 of a treatment period.

In another embodiment, the dosing regimen includes a dosing regimen having a first administration of a therapeutically effective dose of at least one anti-CD40 antibody daily, or on days 1, 3, 5, and 7 of a week in a treatment period; a dosing regimen including a first administration of a therapeutically effective dose of at least one anti-CD40 antibody on days 1 and 3-4 of a week in a treatment period; and a preferred dosing regimen including a first administration of a therapeutically effective dose of at least one anti-CD40 antibody on day 1 of a week in a treatment period. The treatment period may comprise at least 1 week, at least 2 weeks, at least 3 weeks, at least a month, at least 2 months, at least 3 months, at least 6 months, or at least a year. Treatment periods may be subsequent or separated from each other by at least a week, at least 2 weeks, at least a month, at least 3 months, at least 6 months, or at least a year.

In other embodiments, the initial therapeutically effective dose of an anti-CD40 antibody as defined elsewhere herein can be in the lower dosing range (i.e., about 0.3 mg/kg to about 20 mg/kg) with subsequent doses falling within the higher dosing range (i.e., from about 20 mg/kg to about 50 mg/kg).

In alternative embodiments, the initial therapeutically effective dose of an anti-CD40 antibody as defined elsewhere herein can be in the upper dosing range (i e., about 20 mg/kg to about 50 mg/kg) with subsequent doses falling within the lower dosing range (i.e., 0.3 mg/kg to about 20 mg/kg). Thus, in some embodiments of the invention, anti-CD40 antibody therapy may be initiated by administering a “loading dose” of the antibody to the subject in need therapy. By “loading dose” is intended an initial dose of the anti-CD40 antibody that is administered to the subject, where the dose of the antibody administered falls within the higher dosing range (i.e., from about 20 mg/kg to about 50 mg/kg). The “loading dose” can be administered as a single administration, for example, a single infusion where the antibody is administered IV, or as multiple administrations, for example, multiple infusions where the antibody is administered IV, so long as the complete “loading dose” is administered within about a 24-hour period. Following administration of the “loading dose,” the subject is then administered one or more additional therapeutically effective doses of the anti-CD40 antibody. Subsequent therapeutically effective doses can be administered, for example, according to a weekly dosing schedule, or once every two weeks, once every three weeks, or once every four weeks. In such embodiments, the subsequent therapeutically effective doses generally fall within the lower dosing range (i.e., 0.3 mg/kg to about 20 mg/kg).

Alternatively, in some embodiments, following the “loading dose”, the subsequent therapeutically effective doses of the anti-CD40 antibody are administered according to a “maintenance schedule”, wherein the therapeutically effective dose of the antibody is administered once a month, once every 6 weeks, once every two months, once every 10 weeks, once every three months, once every 14 weeks, once every four months, once every 18 weeks, once every five months, once every 22 weeks, once every six months, once every 7 months, once every 8 months, once every 9 months, once every 10 months, once every 11 months, or once every 12 months. In such embodiments, the therapeutically effective doses of the anti-CD40 antibody fall within the lower dosing range (i.e., 0.3 mg/kg to about 20 mg/kg), particularly when the subsequent doses are administered at more frequent intervals, for example, once every two weeks to once every month, or within the higher dosing range (i.e., from about 20 mg/kg to about 50 mg/kg), particularly when the subsequent doses are administered at less frequent intervals, for example, where subsequent doses are administered about one month to about 12 months apart.

The anti-CD40 antibodies present in the pharmaceutical compositions described herein for use in the methods of the invention may be native or obtained by recombinant techniques, and may be from any source, including mammalian sources such as, e.g., mouse, rat, rabbit, primate, pig, and human. Preferably such polypeptides are derived from a human source, and more preferably are recombinant, human proteins from hybridoma cell lines.

Any pharmaceutical composition comprising an anti-CD40 antibody having the binding properties described herein as the therapeutically active component can be used in the methods of the invention. Thus liquid, lyophilized, or spray-dried compositions comprising one or more of the anti-CD40 antibodies may be prepared as an aqueous or nonaqueous solution or suspension for subsequent administration to a subject in accordance with the methods of the invention. Each of these compositions will comprise at least one anti-CD40 antibody as a therapeutically or prophylactically active component. By “therapeutically or prophylactically active component” is intended the anti-CD40 antibody is specifically incorporated into the composition to bring about a desired therapeutic or prophylactic response with regard to treatment, prevention, or diagnosis of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

Formulants may be added to pharmaceutical compositions comprising an anti-CD40 antibody. These formulants may include, but are not limited to, oils, polymers, vitamins, carbohydrates, amine acids, salts, buffers, albumin, surfactants, or bulking agents. Preferably carbohydrates include sugar or sugar alcohols such as mono-, di-, or polysaccharides, or water soluble glucans. The saccharides or glucans can include fructose, glucose, trehalose, mannose, sorbose, xylose, maltose, sucrose, dextran, pullulan, dextrin, α- and β-cyclodextrin, soluble starch, hydroxyethyl starch, and carboxymethylcellulose, or mixtures thereof “Sugar alcohol” is defined as a C₄ to C₈ hydrocarbon having a hydroxyl group and includes galactitol, inositol, mannitol, xylitol, sorbitol, glycerol, and arabitol. These sugars or sugar alcohols may be used individually or in combination. The sugar or sugar alcohol concentration is between 1.0% and 7% w/v., more preferably between 2.0% and 6.0% w/v. Preferably amino acids include levorotary (L) forms of carnitine, arginine, and betaine; however, other amino acids may be added. Preferred polymers include polyvinylpyrrolidone (PVP) with an average molecular weight between 2,000 and 3,000, or polyethylene glycol (PEG) with an average molecular weight between 3,000 and 5,000. Surfactants that can be added to the formulation are shown in EP Nos. 270,799 and 268,110.

The formulants to be incorporated into a pharmaceutical composition should provide for the stability of the anti-CD40 antibody. That is, the anti-CD40 antibody should retain its physical and/or chemical stability and have the desired biological activity, i.e., one or more of the antagonist activities defined herein above.

Methods for monitoring protein stability are well known in the art. See, for example, Jones (1993) Adv. Drug Delivery Rev. 10:29-90; Lee, ed. (1991) Peptide and Protein Drug Delivery (Marcel Dekker, Inc., New York, N.Y.); and the stability assays disclosed herein below. Generally, protein stability is measured at a chosen temperature for a specified period of time. In preferred embodiments, a stable antibody pharmaceutical formulation provides for stability of the anti-CD40 antibody when stored at room temperature (about 25° C.) for at least 1 month, at least 3 months, or at least 6 months, and/or is stable at about 2-8° C. for at least 6 months, at least 9 months, at least 12 months, at least 18 months, at least 24 months.

A protein such as an antibody, when formulated in a pharmaceutical composition, is considered to retain its physical stability at a given point in time if it shows no visual signs (i.e., discoloration or loss of clarity) or measurable signs (for example, using size-exclusion chromatography (SEC) or UV light scattering) of precipitation, aggregation, and/or denaturation in that pharmaceutical composition. With respect to chemical stability, a protein such as an antibody, when formulated in a pharmaceutical composition, is considered to retain its chemical stability at a given point in time if measurements of chemical stability are indicative that the protein (i.e., antibody) retains the biological activity of interest in that pharmaceutical composition. Methods for monitoring changes in chemical stability are well known in the art and include, but are not limited to, methods to detect chemically altered forms of the protein such as result from clipping, using, for example, SDS-PAGE, SEC, and/or matrix-assisted laser desorption ionization/time of flight mass spectrometry; and degradation associated with changes in molecular charge (for example, associated with deamidation), using, for example, ion-exchange chromatography. See, for example, the methods disclosed herein below.

An anti-CD40 antibody, when formulated in a pharmaceutical composition, is considered to retain a desired biological activity at a given point in time if the desired biological activity at that time is within about 30%, preferably within about 20% of the desired biological activity exhibited at the time the pharmaceutical composition was prepared as determined in a suitable assay for the desired biological activity. Assays for measuring the desired biological activity of the anti-CD40 antibodies can be performed as described in the Examples herein. See also the assays described in Schultze et al. (1998) Proc. Natl. Acad. Sci. USA 92:8200-8204; Denton et al. (1998) Pediatr. Transplant. 2:6-15; Evans et al. (2000) J. Immunol. 164:688-697; Noelle (1998) Agents Actions Suppl. 49:17-22; Lederman et al. (1996) Curr Opin. Hematol. 3:77-86; Coligan et al. (1991) Current Protocols in Immunology 13:12; Kwekkeboom et al. (1993) Immunology 79:439-444; and U.S. Pat. Nos. 5,674,492 and 5,847,082.

In some embodiments of the invention, the anti-CD40 antibody is formulated in a liquid pharmaceutical formulation. The anti-CD40 antibody can be prepared using any method known in the art, including those methods disclosed herein above. The anti-CD40 antibody may be recombinantly produced in a CHO cell line.

Where the anti-CD40 antibody is to be stored prior to its formulation, it can be frozen, for example, at ≦−20° C., and then thawed at room temperature for further formulation. The liquid pharmaceutical formulation comprises a therapeutically effective amount of the anti-CD40 antibody. The amount of antibody thereof present in the formulation takes into consideration the route of administration and desired dose volume.

In this manner, the liquid pharmaceutical composition comprises the anti-CD40 antibody at a concentration of about 0.1 mg/ml to about 50.0 mg/ml, about 0.5 mg/ml to about 40.0 mg/ml, about 1.0 mg/ml to about 30.0 mg/ml, about 5.0 mg/ml to about 25.0 mg/ml, about 5.0 mg/ml to about 20.0 mg/ml, or about 15.0 mg/ml to about 25.0 mg/ml. In some embodiments, the liquid pharmaceutical composition comprises the anti-CD40 antibody at a concentration of about 0.1 mg/ml to about 5.0 mg/ml, about 5.0 mg/ml to about 10.0 mg/ml, about 10.0 mg/ml to about 15.0 mg/ml, about 15.0 mg/ml to about 20.0 mg/ml, about 20.0 mg/ml to about 25.0 mg/ml, about 25.0 mg/ml to about 30.0 mg/ml, about 30.0 mg/ml to about 35.0 mg/ml, about 35.0 mg/ml to about 40.0 mg/ml, about 40.0 mg/ml to about 45.0 mg/ml, or about 45.0 mg/ml to about 50.0 mg/ml. In other embodiments, the liquid pharmaceutical composition comprises the anti-CD40 antibody at a concentration of about 15.0 mg/ml, about 16.0 mg/ml, about 17.0 mg/ml, about 18.0 mg/ml, about 19.0 mg/ml, about 20.0 mg/ml, about 21.0 mg/ml, about 22.0 mg/ml, about 23.0 mg/ml, about 24.0 mg/ml, or about 25.0 mg/ml. The liquid pharmaceutical composition comprises the anti-CD40 antibody and a buffer that maintains the pH of the formulation in the range of about pH 5.0 to about pH 7.0, including about pH 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0. In some embodiments, the buffer maintains the pH of the formulation in the range of about pH 5.0 to about pH 6.5, about pH 5.0 to about pH 6.0, about pH 5.0 to about pH 5.5, about pH 5.5 to about 7.0, about pH 5.5 to about pH 6.5, or about pH 5.5 to about pH 6.0.

Any suitable buffer that maintains the pH of the liquid anti-CD40 antibody formulation in the range of about pH 5.0 to about pH 7.0 can be used in the formulation, so long as the physicochemical stability and desired biological activity of the antibody are retained as noted herein above. Suitable buffers include, but are not limited to, conventional acids and salts thereof, where the counter ion can be, for example, sodium, potassium, ammonium, calcium, or magnesium. Examples of conventional acids and salts thereof that can be used to buffer the pharmaceutical liquid formulation include, but are not limited to, succinic acid or succinate, citric acid or citrate, acetic acid or acetate, tartaric acid or tartarate, phosphoric acid or phosphate, gluconic acid or gluconate, glutamic acid or glutamate, aspartic acid or aspartate, maleic acid or maleate, and malic acid or malate buffers. The buffer concentration within the formulation can be from about 1 mM to about 50 mM, including about 1 mM, 2 mM, 5 mM, 8 mM, 10 mM, 15 mM, 20 mM, 25 mM, 30 mM, 35 mM, 40 mM, 45 mM, 50 mM, or other such values within the range of about 1 mM to about 50 mM. In some embodiments, the buffer concentration within the formulation is from about 5 mM to about 15 mM, including about 5 mM, 6 mM, 7 mM, 8 mM, 9 mM, 10 mM, 11 mM, 12 mM, 13 mM, 14 mM, 15 mM, or other such values within the range of about 5 mM to about 15 mM.

In some embodiments of the invention, the liquid pharmaceutical formulation comprises a therapeutically effective amount of the anti-CD40 antibody and succinate buffer or citrate buffer at a concentration that maintains the pH of the formulation in the range of about pH 5.0 to about pH 7.0, preferably about pH 5.0 to about pH 6.5. By “succinate buffer” or “citrate buffer” is intended a buffer comprising a salt of succinic acid or a salt of citric acid, respectively. In a preferred embodiment, the succinate or citrate counterion is the sodium cation, and thus the buffer is sodium succinate or sodium citrate, respectively. However, any cation is expected to be effective. Other possible succinate or citrate cations include, but are not limited to, potassium, ammonium, calcium, and magnesium. As noted above, the succinate or citrate buffer concentration within the formulation can be from about 1 mM to about 50 mM, including about 1 mM, 2 mM, 5 mM, 8 mM, 10 mM, 15 mM, 20 mM, 25 mM, 30 mM, 35 mM, 40 mM, 45 mM, 50 mM, or other such values within the range of about 1 mM to about 50 mM. In some embodiments, the buffer concentration within the formulation is from about 5 mM to about 15 mM, including about 5 mM, 6 mM, 7 mM, 8 mM, 9 mM, 10 mM, 11 mM, 12 mM, 13 mM, 14 mM, or about 15 mM. In other embodiments, the liquid pharmaceutical formulation comprises the anti-CD40 antibody at a concentration of about 0.1 mg/ml to about 50.0 mg/ml, or about 5.0 mg/ml to about 25.0 mg/ml, and succinate or citrate buffer, for example, sodium succinate or sodium citrate buffer, at a concentration of about 1 mM to about 20 mM, about 5 mM to about 15 mM, preferably about 10 mM.

Where it is desirable for the liquid pharmaceutical formulation to be near isotonic, the liquid pharmaceutical formulation comprising the anti-CD40 antibody and a buffer can further comprise an amount of an isotonizing agent sufficient to render the formulation near isotonic. By “near isotonic” is intended the aqueous formulation has an osmolarity of about 240 mmol/kg to about 360 mmol/kg, preferably about 240 to about 340 mmol/kg, more preferably about 250 to about 330 mmol/kg, even more preferably about 260 to about 320 mmol/kg, still more preferably about 270 to about 310 mmol/kg. Methods of determining the isotonicity of a solution are known to those skilled in the art.

Those skilled in the art are familiar with a variety of pharmaceutically acceptable solutes useful in providing isotonicity in pharmaceutical compositions. The isotonizing agent can be any reagent capable of adjusting the osmotic pressure of the liquid pharmaceutical formulation of the present invention to a value nearly equal to that of a body fluid. It is desirable to use a physiologically acceptable isotonizing agent. Thus, the liquid pharmaceutical formulation comprising a therapeutically effective amount of the anti-CD40 antibody and a buffer can further comprise components that can be used to provide isotonicity, for example, sodium chloride; amino acids such as alanine, valine, and glycine; sugars and sugar alcohols (polyols), including, but not limited to, glucose, dextrose, fructose, sucrose, maltose, mannitol, trehalose, glycerol, sorbitol, and xylitol; acetic acid, other organic acids or their salts, and relatively minor amounts of citrates or phosphates. The ordinary skilled person would know of additional agents that are suitable for providing optimal tonicity of the liquid formulation.

In some preferred embodiments, the liquid pharmaceutical formulation comprising an anti-CD40 antibody and a buffer further comprises sodium chloride as the isotonizing agent. The concentration of sodium chloride in the formulation will depend upon the contribution of other components to tonicity. In some embodiments, the concentration of sodium chloride is about 50 mM to about 300 mM, about 50 mM to about 250 mM, about 50 mM to about 200 mM, about 50 mM to about 175 mM, about 50 mM to about 150 mM, about 75 mM to about 175 mM, about 75 mM to about 150 mM, about 100 mM to about 175 mM, about 100 mM to about 200 mM, about 100 mM to about 150 mM, about 125 mM to about 175 mM, about 125 mM to about 150 mM, about 130 mM to about 170 mM, about 130 mM to about 160 mM, about 135 mM to about 155 mM, about 140 mM to about 155 mM, or about 145 mM to about 155 mM. In one such embodiment, the concentration of sodium chloride is about 150 mM. In other such embodiments, the concentration of sodium chloride is about 150 mM, the buffer is sodium succinate or sodium citrate buffer at a concentration of about 5 mM to about 15 mM, the liquid pharmaceutical formulation comprises a therapeutically effective amount of the anti-CD40 antibody and the formulation has a pH of about pH 5.0 to about pH 7.0, about pH 5.0 to about pH 6.0, or about pH 5.5 to about pH 6.5. In other embodiments, the liquid pharmaceutical formulation comprises the anti-CD40 antibody at a concentration of about 0.1 mg/ml to about 50.0 mg/ml or about 5.0 mg/ml to about 25.0 mg/ml, about 150 mM sodium chloride, and about 10 mM sodium succinate or sodium citrate, at a pH of about pH 5.5.

Protein degradation due to freeze thawing or mechanical shearing during processing of a liquid pharmaceutical formulation of the present invention can be inhibited by incorporation of surfactants into the formulation in order to lower the surface tension at the solution-air interface. Thus, in some embodiments, the liquid pharmaceutical formulation comprises a therapeutically effective amount of the anti-CD40 antibody, a buffer, and further comprises a surfactant. In other embodiments, the liquid pharmaceutical formulation comprises an anti-CD40 antibody, a buffer, an isotonizing agent, and further comprises a surfactant.

Typical surfactants employed are nonionic surfactants, including polyoxyethylene sorbitol esters such as polysorbate 80 (Tween 80) and polysorbate 20 (Tween 20); polyoxypropylene-polyoxyethylene esters such as Pluronic F68; polyoxyethylene alcohols such as Brij 35; simethicone; polyethylene glycol such as PEG400; lysophosphatidylcholine; and polyoxyethylene-p-t-octylphenol such as Triton X-100. Classic stabilization of pharmaceuticals by surfactants or emulsifiers is described, for example, in Levine et al. (1991) J. Parenteral Sci. Technol. 45(3):160-165. A preferred surfactant employed in the practice of the present invention is polysorbate 80. Where a surfactant is included, it is typically added in an amount from about 0.001% to about 1.0% (w/v), about 0.001% to about 0.5%, about 0.001% to about 0.4%, about 0.001% to about 0.3%, about 0.001% to about 0.2%, about 0.005% to about 0.5%, about 0.005% to about 0.2%, about 0.01% to about 0.5%, about 0.01% to about 0.2%, about 0.03% to about 0.5%, about 0.03% to about 0.3%, about 0.05% to about 0.5%, or about 0.05% to about 0.2%.

Thus, in some embodiments, the liquid pharmaceutical formulation comprises a therapeutically effective amount of the anti-CD40 antibody, the buffer is sodium succinate or sodium citrate buffer at a concentration of about 1 mM to about 50 mM, about 5 mM to about 25 mM, or about 5 mM to about 15 mM; the formulation has a pH of about pH 5.0 to about pH 7.0, about pH 5.0 to about pH 6.0, or about pH 5.5 to about pH 6.5; and the formulation further comprises a surfactant, for example, polysorbate 80, in an amount from about 0.001% to about 1.0% or about 0.001% to about 0.5%. Such formulations can optionally comprise an isotonizing agent, such as sodium chloride at a concentration of about 50 mM to about 300 mM, about 50 mM to about 200 mM, or about 50 mM to about 150 mM. In other embodiments, the liquid pharmaceutical formulation comprises the anti-CD40 antibody at a concentration of about 0.1 mg/ml to about 50.0 mg/ml or about 5.0 mg/ml to about 25.0 mg/ml, including about 20.0 mg/ml; about 50 mM to about 200 mM sodium chloride, including about 150 mM sodium chloride; sodium succinate or sodium citrate at about 5 mM to about 20 mM, including about 10 mM sodium succinate or sodium citrate; sodium chloride at a concentration of about 50 mM to about 200 mM, including about 150 mM; and optionally a surfactant, for example, polysorbate 80, in an amount from about 0.001% to about 1.0%, including about 0.001% to about 0.5%; where the liquid pharmaceutical formulation has a pH of about pH 5.0 to about pH 7.0, about pH 5.0 to about pH 6.0, about pH 5.0 to about pH 5.5, about pH 5.5 to about pH 6.5, or about pH 5.5 to about pH 6.0.

The liquid pharmaceutical formulation can be essentially free of any preservatives and other carriers, excipients, or stabilizers noted herein above. Alternatively, the formulation can include one or more preservatives, for example, antibacterial agents, pharmaceutically acceptable carriers, excipients, or stabilizers described herein above provided they do not adversely affect the physicochemical stability of the anti-CD40 antibody. Examples of acceptable carriers, excipients, and stabilizers include, but are not limited to, additional buffering agents, co-solvents, surfactants, antioxidants including ascorbic acid and methionine, chelating agents such as EDTA, metal complexes (for example, Zn-protein complexes), and biodegradable polymers such as polyesters. A thorough discussion of formulation and selection of pharmaceutically acceptable carriers, stabilizers, and isomolytes can be found in Remington: The Science and Practice of Pharmacy (21st edition, Lippincott Williams & Wilkins, May 2005).

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, succinate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG), and Pluronics.

After the liquid pharmaceutical formulation or other pharmaceutical composition described herein is prepared, it can be lyophilized to prevent degradation. Methods for lyophilizing liquid compositions are known to those of ordinary skill in the art. Just prior to use, the composition may be reconstituted with a sterile diluent (Ringer's solution, distilled water, or sterile saline, for example) that may include additional ingredients. Upon reconstitution, the composition is preferably administered to subjects using those methods that are known to those skilled in the art.

The anti-CD40 antibody-containing pharmaceutical composition may be a composition as described in co-owned International Patent Application No. PCT/US2007/066757 published as WO 2007/124299. In particular, a pharmaceutical composition for use in the combination therapy of the invention may comprise (i) an anti-CD40 antibody, a buffering agent to maintain the pH of the composition between around pH 5.0 and pH 7.0, and (iii) an amount of arginine-HCl sufficient to render the liquid composition near isotonic. In these compositions, the buffering agent may be a citrate/citric acid buffer. The composition may further comprise a non-ionic surfactant and/or L-methionine as further stabilizing agents. The composition may have an osmolarity of about 240 mmol/kg to about 360 mmol/kg. The concentration of the buffering agent may be from about 5 mM to about 100 mM, from about 5 mM to about 20 mM, or from about 5 mM to about 15 mM (e.g., 10 mM). The composition may have a pH of from pH 5.0 to pH 6.0 (e.g., around pH 5.5). The composition may comprise arginine-HCl at a concentration of about 50 mM to about 200 mM, or from about 100 mM to about 175 mM (e.g., about 150 mM). The composition may further comprise the surfactant polysorbate, for example at a concentration of about 0.001% to about 1.0% (w/v), or at a concentration of about 0.025% to about 0.1% (w/v). The composition may further comprise methionine at a concentration of about 0.5 mM to about 20.0 mM or at a concentration of about 1.0 mM to about 20.0 mM (e.g., about 5.0 mM). The anti-CD40 antibody may be present in the composition at about 0.1 mg/ml to about 50.0 mg/ml, or at about 1.0 mg/ml to about 35.0 mg/ml, or at about 10.0 mg/ml to about 35.0 mg/ml.

The invention also involves use of the chemotherapeutic agents cyclophosphamide (brand name Cytoxan), doxorubicin (brand name Adriamycin), vincristine (brand name Oncovin) and prednisone (brand name Deltasone). The use of these four chemotherapeutic agents in combination is referred to as CHOP. CHOP regimens are commonly used to treat patients with non-Hodgkin's lymphoma, and CHOP has been considered the standard therapy for patients with diffuse large B-cell lymphoma (DLBCL) for more than twenty-five years (Feugier et al. (2005) J. Clin. Oncol. 23(18):4117-4126; Habermann et al. (2006) J. Clin. Oncol. 24(19): 3121-3127). CHOP has been used in combination with rituximab in the treatment of DLBCL (Feugier et al. (2005) J. Clin. Oncol. 23(18):4117-4126; Habermann et al. (2006) J. Clin. Oncol. 24(19):3121-3127).

CHOP is a combination of three chemotherapy drugs (cyclophosphamide, doxorubicin and vincristine) and a steroid (prednisone). CHOP chemotherapy is associated with numerous side effects, the most common being fatigue, reduced blood cell counts due to effects on bone marrow, nausea, hair loss, infertility, mouth sores and ulcers, loss of appetite and nervous system symptoms (e.g., pins and needles or abdominal pain). The methods of the invention may allow one or more of these side effects to be reduced or eliminated, by allowing lower dose CHOP regimens to be used. Accordingly, in some embodiments the methods, uses, compositions and kits of the invention may be used for treating a human patient for a disease or condition associated with neoplastic B-cell growth, whilst avoiding or reducing one or more of the side effects normally associated with administration of CHOP.

The combination therapy of the invention may also allow one or more side effects associated with administration of anti-CD40 antibodies to be reduced or eliminated, by allowing lower doses of anti-CD40 antibodies to be used. Accordingly, in some embodiments the methods, uses, compositions and kits of the invention may be used for treating a human patient for a disease or condition associated with neoplastic B-cell growth, whilst avoiding or reducing one or more of the side effects normally associated with administration of an anti-CD40 antibody.

Any pharmaceutical compositions comprising the CHOP components as the therapeutically active component(s) can be used in the methods of the invention. These will contain one or more of the CHOP components and a pharmaceutically acceptable carrier or excipient, e.g., a pharmaceutically acceptable carrier or excipient as described elsewhere herein. Suitable pharmaceutical compositions are well known in the art. By “therapeutically active component” is intended that the relevant therapeutic agent(s) are specifically incorporated into the composition to bring about a desired therapeutic response with regard to treatment of a disease or condition within a subject when the pharmaceutical composition is administered to that subject. The CHOP components are administered at concentrations that are “therapeutically effective” to treat a disease or condition associated with neoplastic B-cell growth.

The CHOP components may be administered by any appropriate route of administration. Cyclophosphamide, doxorubicin and vincristine are normally administered intravenously, whereas prednisone is normally administered orally. Methods to accomplish this administration are known to those of ordinary skill in the art.

CHOP is normally administered in cycles of treatment, each cycle comprising administration of cyclophosphamide at 750 mg/m² on day 1, doxorubicin at 50 mg/m² on day 1, vincristine at 1.4 mg/m² on day 1, and prednisone at 100 mg/m² on days 1 through 5. The cycle is generally repeated every three weeks (21 days). A usual course of treatment consists of six to eight cycles in total.

In the methods, uses, compositions and kits of the invention the cyclophosphamide may be used at 75-1000 mg/m², or at 185-1000 mg/m², or at 500-1000 mg/m², or at 700-800 mg/m² (e.g., at 750 mg/m²). The doxorubicin may be used at 5-70 mg/m², or at 12-70 mg/m², or at 35-70 mg/m² or at 45-55 mg/m² (e.g., at 50 mg/m²). The vincristine may be used at 0.1-2.0 mg/m², or at 0.7-2.0 mg/m², or at 1.0-2.0 mg/m², or at 1.0-1.6 mg/m² (e.g., at 1.4 mg/m²). The prednisone may be used at 10-130 mg/m², or at 50-130 mg/m², or at at 65-130 mg/m², or at 85-125 mg/m² (e.g., at 100 mg/m²). The skilled person will readily be able to select an appropriate CHOP regimen for use in the combination therapy of the invention.

In the methods, uses, compositions and kits of the invention the CHOP regimen will preferably be repeated every three weeks, but may be repeated every four weeks, every five weeks, every six weeks, every seven weeks, every eight weeks, every nine weeks, or every ten weeks, if desired. The combination therapy of the invention may enable lower doses of CHOP to be used whilst retaining therapeutic efficacy, thereby allowing the CHOP regimen to be repeated more frequently, such as every week or every two weeks. The CHOP can be administered for any desired number of cycles, e.g., 1-20 cycles, preferably 3-15 cyles, more preferably 5-10 cycles.

The term “comprising” encompasses “including” as well as “consisting”. For example, a composition “comprising” X may consist exclusively of X or may include something additional, e.g., X+Y.

The word “substantially” does not exclude “completely” e.g., a composition which is “substantially free” from Y may be completely free from Y. Where necessary, the word “substantially” may be omitted from the definition of the invention.

The term “about” in relation to a numerical value x means, for example, x±10%. Various aspects and embodiments of the present invention will now be described in more detail by way of example only. It will be appreciated that modification of detail may be made without departing from the scope of the invention.

### EXPERIMENTAL

The anti-CD40 antibody used in the examples below is the monoclonal antibody HCD122 (formerly known as CHIR-12.12). The production, sequencing and characterisation of HCD122 has already been described.

### Example 1

### Anti-Tumor Activity of HCD122 in Combination with CHOP (H-CHOP)

The human monoclonal antibody HCD122 and CHOP have each shown anti-tumor efficacy in RL and SU-DHL-4 lymphoma models when used alone. The RL cell line (ATCC; CRL-2261) is a human B cell lymphoma cell line established from a 52 year old Caucasian male patient with NHL. The SU-DHL-4 cell line (DSMZ; ACC 495) is a human B cell lymphoma cell line established from the peritoneal effusion of a 38 year old man with B-NHL (diffuse large cell, cleaved cell type). These cells lines are both reported to be negative for the Epstein-Barr virus genome, in contrast to many of the common lymphoma cell lines used in the field. The use of cell lines that are positive for the Epstein-Barr virus may lead to problems when interpreting experimental data, due to influences on signalling by the oncogenic EBV in those cell lines. The RL and SU-DHL-4 lymphoma cell lines were specifically chosen by the inventors because they are EBV negative, which allows greater confidence that the results are indeed authentic.

The activity of HCD122 in combination with CHOP was evaluated in the RL diffuse large B-cell lymphoma (DLBCL) xenograft model, and compared to the activities of HCD122 alone and CHOP alone. The combination of HCD122 and CHOP is referred to below as H-CHOP. The therapeutic efficacy of H-CHOP was also compared to the known combination of CHOP with the chimeric anti-CD20 monoclonal antibody rituximab, commonly referred to as R-CHOP.

### Materials and Methods

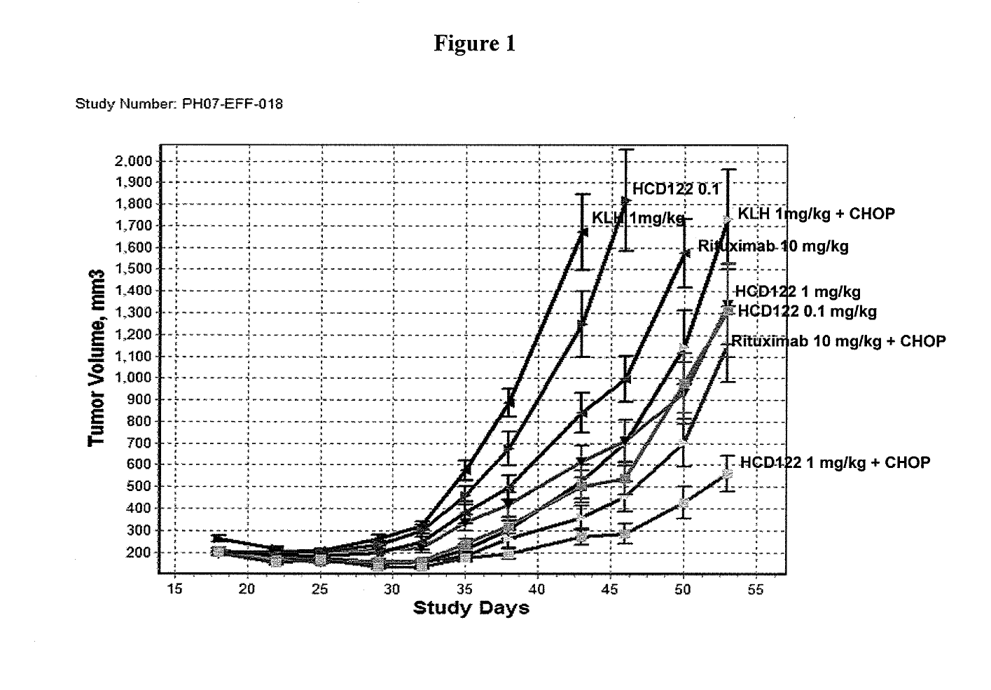

The anti-tumor activity of HCD122 was tested in RL DLBCL xenograft models in combination with CHOP in CB17/SCID mice. 10×10⁶ RL cells were subcutaneously implanted with equal volume of Matrigel™ in the animals' midline thoracic vertebral region in a 200 μl volume. The antibody administration was initiated when the mean tumor volume was 150-200 mm³ in size (noted as day 1 in FIG. 1). HCD122, rituximab and the negative control human IgG1 antibody were each administered by intraperitoneal injection. All monoclonal antibodies were administered on a once-a-week schedule, and the length of treatment was 4 weeks. The CHOP regimen was administered at these doses and schedule: prednisone, 0.2 mg/kg p.o. days 1-5; cytoxan, 40 mg/kg, i.v., day 1; doxorubicin 3.3 mg/kg, i.v., day 1; vincristine, 0.5 mg/kg, i.v., day 1. Group size was n=12. For tumor volume measurements, length then width were measured with a digital caliper. The measurements were recorded twice a week once the tumors became visible. Tumor volumes and doubling times were calculated based on the formula, Volume=L×W²/2. The animals' weights were recorded and evaluated as a per group average.

### Results

The results of these experiments are shown in FIG. 1 and Tables 1 and 2 below.

TABLE 1
Tumor Growth Inhibition on Day 25

| | TGI | p value |
|---|---|---|
| Control huIgG (1 mg/kg) | 0 | — |
| HCD122 (0.1 mg/kg) | 28.65% | p > 0.05 |
| Rituximab (10 mg/kg) | 56.06% | p < 0.001 |
| HCD122 (1 mg/kg) | 71.15% | p < 0.001 |
| CHOP | 77.96% | p < 0.001 |
| HCD122 (0.1 mg/kg) + CHOP | 79.94% | p < 0.001 |
| Rituximab (10 mg/kg) + CHOP | 90.07% | p < 0.001 |
| HCD122 (1 mg/kg) + CHOP | 95.18% | p < 0.001 |

All of the therapies significantly reduced tumor growth at day 25 when compared to treatment with huIgG1 control antibody. The observed tumor growth inhibition (TGI) with CHOP alone or HCD122 alone (at 1 mg/kg) was 77% and 71%, respectively (p<0.001; Tukey test). In contrast, the observed TGI for the H-CHOP combination (using HCD122 at 1 mg/kg) was 95% (p<0.001; Tukey test). The observed TGI for the R-CHOP combination (using rituximab at 10 mg/kg) was 90% (p<0.001; Tukey test).

TABLE 2
Tumor Growth Delay

| | Tumor-growth delay (days) |
|---|---|
| KLH (1 mg/kg) | 0 |
| Rituximab (10 mg/kg) | 4 |
| CHOP | 8 |
| Rituximab (10 mg/kg) + CHOP | 12.5 |
| KLH (1 mg/kg) | 0 |
| HCD122 (0.1 mg/kg) | 1.5 |
| CHOP | 8 |
| HCD122 (0.1 mg/kg + CHOP) | 9 |
| Control huIgG (1 mg/kg) | 0 |
| HCD122 (1 mg/kg) | 6 |
| CHOP | 8 |
| HCD122 (1 mg/kg) + CHOP | 17.5 |

Tumor growth delay (time to reach tumor size of 500 mm³) was significantly longer for H-CHOP (17.5 days), than for CHOP alone (8 days) or HCD122 alone (6 days) (p<0.001). No toxicity was observed with the H-CHOP combination. At the end of the study (day 35) reduction in tumor growth was significantly greater in the treatment group that received H-CHOP (1 mg/kg HCD122) than the groups that received R-CHOP (10 mg/kg rituximab, p<0.05; Tukey test) or CHOP alone (p<0.001; Tukey test).

These data show that treatment with the H-CHOP combination results in greater anti-tumor efficacy than treatment with either HCD122 alone or CHOP alone. When HCD122 was used at 1 mg/kg, the H-CHOP combination provided greater therapeutic efficacy than would be expected if the effects of each agent were merely additive, i.e., the H-CHOP combination was found to provide a synergistic therapeutic effect. These data therefore suggest that the H-CHOP combination can be used to improve anti-tumor therapy in human patients that might otherwise have been treated with CHOP alone or HCD122 alone, e.g., by providing an enhanced therapeutic effect or by allowing a reduction in CHOP dosages to reduce or eliminate one or more of the side-effects associated with administration of CHOP.

These data also show that treatment with the H-CHOP combination results in greater anti-tumor efficacy than treatment with either rituximab alone or with the known combination of rituximab and CHOP (R-CHOP). These data therefore suggest that the H-CHOP combination can be used to improve anti-tumor therapy in human patients that might otherwise have been treated with rituximab alone or with R-CHOP, e.g., by providing an enhanced therapeutic effect or by allowing a reduction in CHOP dosages to reduce or eliminate one or more of the side-effects associated with CHOP.

### Example 2

### HCD122 Reverses CD40L-Induced Resistance to CHOP

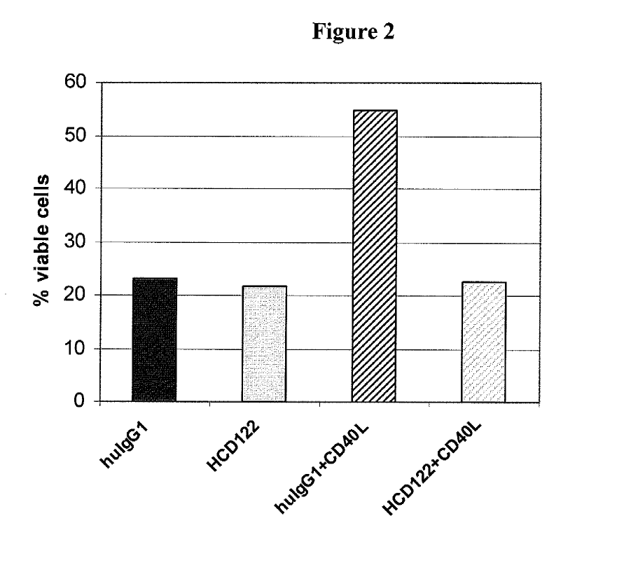

Experiments were performed to elucidate the mechanism by which the H-CHOP combination provides unexpectedly potent anti-tumour efficacy in vivo. SU-DHL-4 cells were cultured in the presence of (i) negative control huIgG1 antibody, (ii) HCD122, (iii) huIgG1 and CD40L or (iv) HCD122 and CD40L. SU-DHL-4 cells were seeded at 30,000 cells/well. The antibodies were all used at 10 μg/ml. Recombinant human soluble CD40L was used at 1 μg/ml with ligand enhancer at 2 μg/ml. All cells were treated with cytoxan at 1 mg/ml, prednisone at 15 μg/ml, doxrubicin at 2.5 ng/ml and vincristin at 1 pg/ml. Cells were cultured for 3 days and the percentage of viable cells determined using CellTiter-Glo. The results of these experiments are shown in FIG. 2. These data show that CD40L induces resistance to CHOP cytotoxicity against SU-DHL-4 cells, but that this resistance can be overcome by using HCD122, thereby allowing the CHOP to have its full cytotoxic effect. These data help to explain the unexpectedly potent anti-tumour efficacy of the H-CHOP.

### Example 3

### Effect of HCD122 on Activation of NFkB

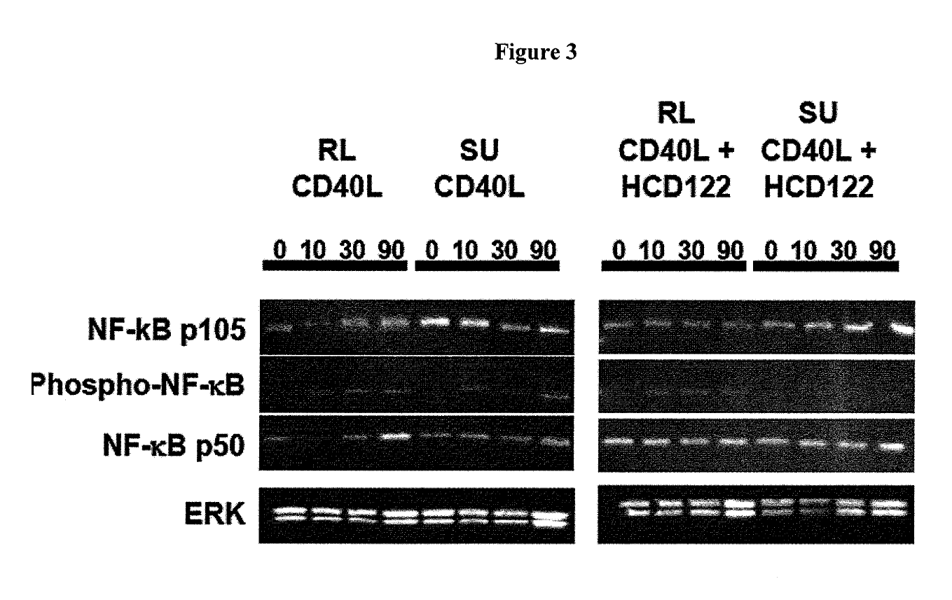

RL and SU-DHL-4 cells were stimulated with CD40L for 0, 10, 30, and 90 minutes and Western blots were performed (FIG. 3). It was found that phosphorylation of p65 was induced within minutes of stimulating the RL or SU-DHL-4 cells with CD40L. The phosphorylation persisted in these cell lines for at least 90 minutes. In addition, it was found that phosphorylation of p65 in both the RL and SU-DHL-4 cells stimulated with CD40L in the presence of HCD122 was greatly inhibited (FIG. 3). These data demonstrate that NF-kB activation induced by CD40L is completely blocked by HCD122 in both RL and SU-DHL-4 cells. Down-regulating NF-kB activation in a cell may sensitize the cell to CHOP cytotoxicity (Chuang et al. (2002) Biochemical Pharmacology 63:1709-1716; Cheng et al. (2000) Oncogene 19:4936-4940). These data showing that HCD122 down-regulates NF-kB activation therefore help to explain why CD40L-induced resistance to CHOP cytotoxicity can be overcome using HCD122.

### Example 4

### Effect of HCD122 on Cell-Surface Adhesion Molecules

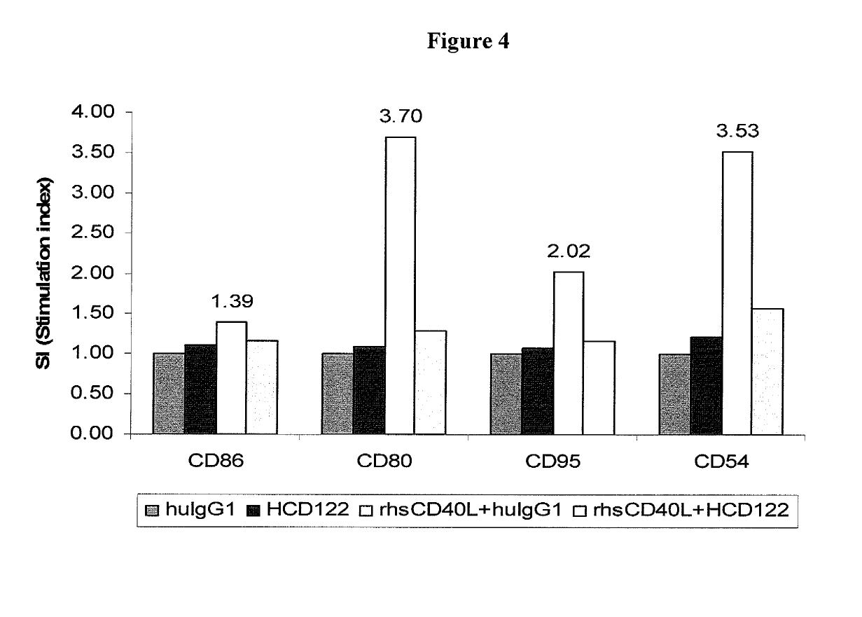

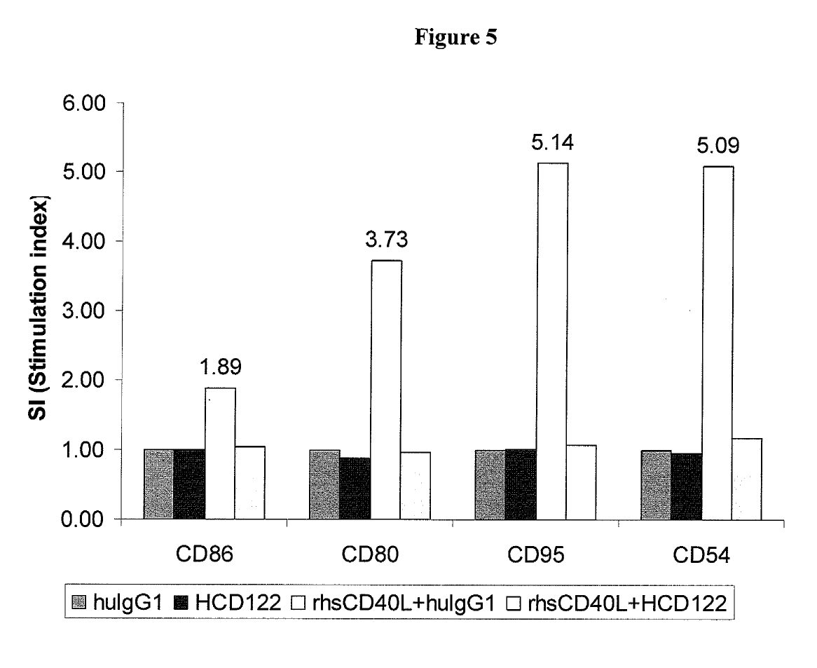

To further elucidate the mechanism by which the H-CHOP combination results in unexpectedly potent anti-tumour efficacy in vivo, further experiments were performed. The ability of B-cells cells to aggregate and interact with their microenvironment may affect the efficacy of therapeutics. The effects of HCD122 on the expression of adhesion molecules in the RL and SU-DHL-4 cell lines was therefore examined. In these studies, HCD122 was found to inhibit CD40L-induced expression of CD54, CD86 and CD95 in both the RL and SU-DHL-4 cell lines. The results for the RL cell line are shown in FIG. 4. The results for the SU-DHL-4 cell lines are shown in FIG. 5.

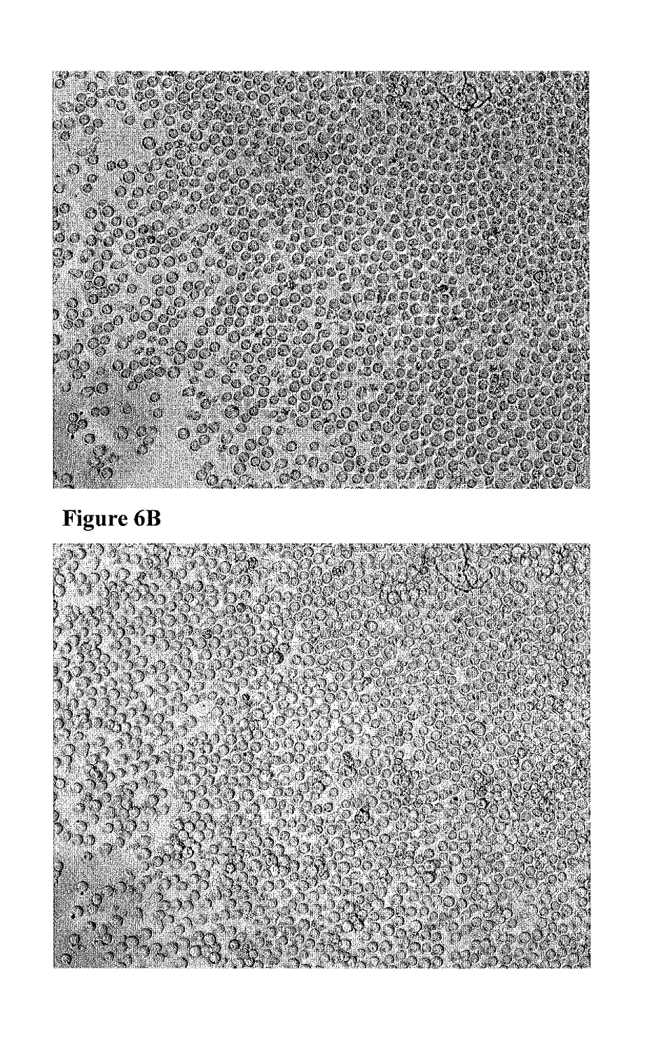

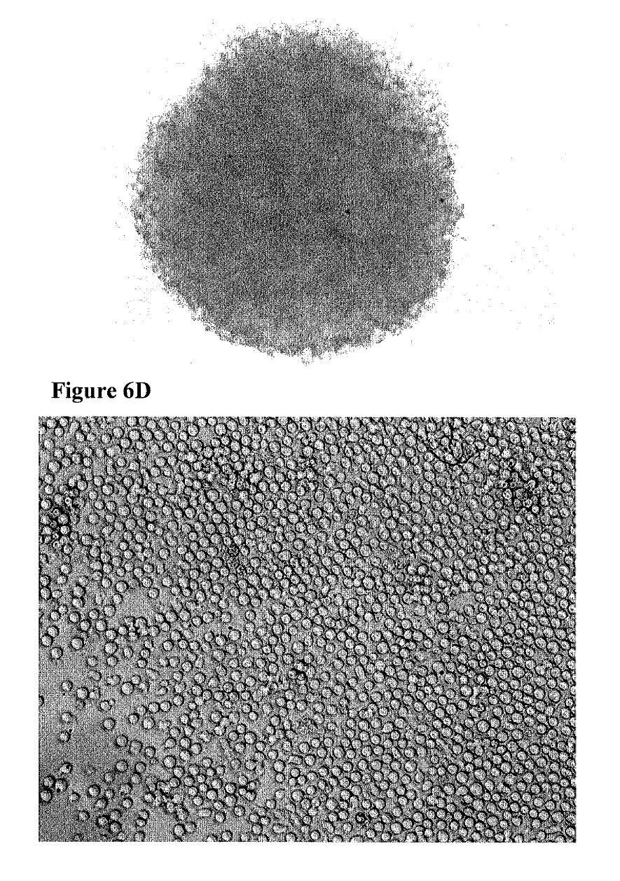

The effect of HCD122 on CD40L-induced aggregation of SU-DHL-4 cells was analysed by microscopy and it was found that HCD122 inhibited this aggregation. The results of these experiments are shown in FIGS. 6A-6D. FIG. 6A shows cells treated with huIgG1. FIG. 6B shows cells treated with HCD122. FIG. 6C shows cells treated with huIgG1 and CD40L. FIG. 6D shows cells treated with HCD122 and CD40L.

These data suggest that CD40L may reduce the efficacy of therapeutics such as CHOP in vivo by causing B-cells to aggregate, and that this aggregation can be prevented using HCD122. These data help to further explain why the H-CHOP combination results in unexpectedly potent anti-tumour efficacy in vivo.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the list of embodiments and appended claims. Although specific terms are employed herein, they are used in a generic and descriptive sense only and not for purposes of limitation.

All publications and patent applications cited herein are incorporated in full by reference to the same extent as if each individual publication or patent application was specifically and individually indicated to be incorporated by reference.

1. A method for treating a human patient for a disease or condition associated with neoplastic B-cell growth, said method comprising administering to said patient cyclophosphamide, doxorubicin, vincristine and prednisone (CHOP) in combination with an anti-CD40 antibody, wherein said anti-CD40 antibody is free of significant agonist activity when bound to CD40 antigen on the surface of human B-cells, and wherein said patient has previously been administered (i) CHOP, (ii) the chimeric anti-CD20 monoclonal antibody rituximab, or (iii) combination therapy with CHOP and rituximab.

2. A method according to claim 1, wherein said disease or condition is refractory to therapy with (i) CHOP, (ii) the chimeric anti-CD20 monoclonal antibody rituximab, or (iii) combination therapy with CHOP and rituximab.

3. A method according to claim 1, wherein said patient has relapsed after therapy with (i) CHOP, (ii) the chimeric anti-CD20 monoclonal antibody rituximab, or (iii) combination therapy with CHOP and rituximab.

4. A method according to claim 1, wherein the CHOP and the anti-CD40 antibody are administered to the patient at the same time.

5. A method according to claim 1, wherein the CHOP and the anti-CD40 antibody are administered to the patient sequentially.

6. A method according to claim 5, wherein a first cycle of CHOP is administered to the patient before a first dose of an anti-CD40 antibody is administered to the patient.

7. A method according to claim 5, wherein a first cycle of CHOP is administered to the patient after a first dose of an anti-CD40 antibody is administered to the patient.

8-11. (canceled)

12. A method according to claim 1, wherein said disease or condition is selected from the group consisting of acute lymphoblastic leukemia (ALL), acute myelogenous leukemia (AML), chronic myelogenous leukemia (CML), chronic lymphocytic leukemia (CLL), prolymphocytic leukemia (PLL), small lymphocytic leukemia (SLL), diffuse small lymphocytic leukemia (DSLL), diffuse large B-cell lymphoma (DLBCL), hairy cell leukemia, non-Hodgkin's lymphomas, Hodgkin's disease, Epstein-Barr Virus (EBV) induced lymphomas, myelomas such as multiple myeloma, Waldenstrom's macroglobulinemia, heavy chain disease, mucosal associated lymphoid tissue lymphoma, monocytoid B cell lymphoma, splenic lymphoma, lymphomatoid granulomatosis, intravascular lymphomatosis, immunoblastic lymphomas, and AIDS-related lymphomas.

13. A method according to claim 12, wherein said disease or condition is a non-Hodgkin's lymphoma.

14. A method according to claim 13, wherein said non-Hodgkin's lymphoma is diffuse large B-cell lymphoma (DLBCL).

15. A method according to claim 1, wherein said anti-CD40 antibody is a monoclonal antibody that binds domain 2 of human CD40 antigen.

16. A method according to claim 1, wherein said anti-CD40 antibody is a monoclonal antibody that binds to an epitope comprising residues 82-87 of the human CD40 sequence shown in SEQ ID NO:7 or SEQ ID NO:9.

17. A method, according to claim 1, wherein said anti-CD40 antibody is selected from the group consisting of:
a) the monoclonal antibody HCD122, produced by the hybridoma cell line deposited with the ATCC as Patent Deposit No. PTA-5543;
b) an antibody comprising an amino acid sequence selected from the group consisting of the sequence shown in SEQ ID NO:2, the sequence shown in SEQ ID NO:4, the sequence shown in SEQ ID NO:5, both the sequences shown in SEQ ID NO:2 and SEQ ID NO:4, and both the sequences shown in SEQ ID NO:2 and SEQ ID NO:5;
c) an antibody comprising an amino acid sequence selected from the group consisting of the sequence shown in SEQ ID NO:17, the sequence shown in SEQ ID NO:19, the sequence shown in SEQ ID NO:20, both the sequences shown in SEQ ID NO:17 and SEQ ID NO:19, and both the sequences shown in SEQ ID NO:17 and SEQ ID NO:20;
d) an antibody comprising an amino acid sequence selected from the group consisting of the sequence shown in SEQ ID NO:16, the sequence shown in SEQ ID NO:18, and both the sequences shown in SEQ ID NO:16 and SEQ ID NO:18;
e) an antibody having an amino acid sequence encoded by a nucleic acid molecule comprising a nucleotide sequence selected from the group consisting of the sequence shown in SEQ ID NO:1, the sequence shown in SEQ ID NO:3, and both the sequences shown in SEQ ID NO:1 and SEQ ID NO:3;
f) an antibody having a light chain variable domain (V_{L}) that comprises the amino acid sequence as shown in SEQ ID NO:10 for CDR-L1, the amino acid sequence as shown in SEQ ID NO:11 for CDR-L2, and the amino acid sequence as shown in SEQ ID NO:12 for CDR-L3;
g) an antibody having a heavy chain variable domain (V_{H}) that comprises the amino acid sequence as shown in SEQ ID NO:13 for CDR-H1, the amino acid sequence as shown in SEQ ID NO:14 for CDR-H2, and the amino acid sequence as shown in SEQ ID NO:15 for CDR-H3; and
h) an antibody having a light chain variable domain (V_{L}) that comprises the amino acid sequence as shown in SEQ ID NO:10 for CDR-L1, the amino acid sequence as shown in SEQ ID NO:11 for CDR-L2, and the amino acid sequence as shown in SEQ ID NO:12 for CDR-L3, and having a heavy chain variable domain (V_{H}) that comprises the amino acid sequence as shown in SEQ ID NO:13 for CDR-H1, the amino acid sequence as shown in SEQ ID NO:14 for CDR-H2, and the amino acid sequence as shown in SEQ ID NO:15 for CDR-H3.

18. A method according to claim 1, wherein said anti-CD40 antibody is obtained from a CHO cell containing one or more expression vectors encoding the antibody.

19. A method according to claim 1, wherein said anti-CD40 antibody is the monoclonal antibody HCD122 (CHIR-12.12) produced by the hybridoma cell line deposited with the ATCC as Patent Deposit No. PTA-5543.

20. A method according to claim 1, wherein said anti-CD40 antibody is an antigen-binding antibody fragment selected from the group consisting of a Fab fragment, a F(ab′)₂ fragment, and a Fv fragment, wherein the fragment is free of significant agonist activity when bound to CD40 antigen on the surface of human B-cells.

21. A method for preventing or reducing resistance to CHOP cytotoxicity in neoplastic human B-cells, comprising the step of contacting one or more neoplastic human B-cells with an anti-CD40 antibody, wherein said anti-CD40 antibody is free of significant agonist activity when bound to CD40 antigen on the surface of human B-cells.

22. A method for preventing or reducing B-cell resistance to CHOP cytotoxicity in a human patient, comprising the step of administering to said patient an anti-CD40 antibody, wherein said anti-CD40 antibody is free of significant agonist activity when bound to CD40 antigen on the surface of human B-cells.

23. A method according to claim 22, wherein the anti-CD40 antibody down-regulates the NF-kB activation in B-cells that is induced by CD40 signalling and which contributes to the development of B-cell resistance to CHOP cytotoxicity.

24. A method according to claim 22, wherein the anti-CD40 antibody inhibits the expression of one or more cell-surface adhesion molecules on B-cells that is induced by CD40 signalling and which contribute(s) to the development of B-cell resistance to CHOP cytotoxicity.

25-26. (canceled)


SEQUENCE LISTING (20 sequences)

SEQ ID NO: 1 - DNA, 720 bp, Artificial Sequence
  Coding sequence for light chain of HCD122 human anti-CD40 antibody
atggcgctccctgctcagctcctggggctgctaatgctctgggtctctggatccagtggggatattgtgatgactcagtctccactctccctgaccgtcacccctggagagccggcctccatctcctgcaggtccagtcagagcctcctgtatagtaatggatacaactatttggattggtacctgcagaagccagggcagtctccacaggtcctgatctctttgggttctaatcgggcctccggggtccctgacaggttcagtggcagtggatcaggcacagattttacactgaaaatcagcagagtggaggctgaggatgttggggtttattactgcatgcaagctcgacaaactccattcactttcggccctgggaccaaagtggatatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - protein, 239 aa, Artificial Sequence
  Light chain of HCD122 human anti-CD40 antibody
MALPAQLLGLLMLWVSGSSGDIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 2016 bp, Artificial Sequence
  Coding sequence for heavy chain of HCD122 human anti-CD40 antibody (with introns)
atggagtttgggctgagctgggttttccttgttgctattttaagaggtgtccagtgtcaggtgcagttggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgaggaaagtaatagataccatgcagactccgtgaagggccgattcaccatctccagagacaattccaagatcacgctgtatctgcaaatgaacagcctcagaactgaggacacggctgtgtattactgtgcgagagatgggggtatagcagcacctgggcctgactactggggccagggaaccctggtcaccgtctcctcagcaagtaccaagggcccatccgtcttccccctggcgcccgctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 469 aa, Artificial Sequence
  Heavy chain of HCD122 human anti-CD40 antibody
MEFGLSWVFLVAILRGVQCQVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPASKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 469 aa, Artificial Sequence
  Heavy chain of variant of HCD122 human anti-CD40 antibody
MEFGLSWVFLVAILRGVQCQVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 612 bp, Homo sapiens
  CDS (1)...(612)
  Coding sequence for short isoform of human CD40
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaaggtccccaggatcggctgagagccctggtggtgatccccatcatcttcgggatcctgtttgccatcctcttggtgctggtctttatcaaaaaggtggccaagaagccaaccaataa

SEQ ID NO: 7 - protein, 203 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTRSPGSAESPGGDPHHLRDPVCHPLGAGLYQKGGQEANQ

SEQ ID NO: 8 - DNA, 834 bp, Homo sapiens
  CDS (1)...(834)
  Coding sequence for long isoform of human CD40
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 9 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 10 - protein, 16 aa, Artificial Sequence
  HCD122 light chain CDR1
RSSQSLLYSNGYNYLD

SEQ ID NO: 11 - protein, 7 aa, Artificial Sequence
  HCD122 light chain CDR2
LGSNRAS

SEQ ID NO: 12 - protein, 9 aa, Artificial Sequence
  HCD122 light chain CDR3
MQARQTPFT

SEQ ID NO: 13 - protein, 10 aa, Artificial Sequence
  HCD122 heavy chain CDR1
GFTFSSYGMH

SEQ ID NO: 14 - protein, 17 aa, Artificial Sequence
  HCD122 heavy chain CDR2
VISYEESNRYHADSVKG

SEQ ID NO: 15 - protein, 11 aa, Artificial Sequence
  HCD122 heavy chain CDR3
DGGIAAPGPDY

SEQ ID NO: 16 - protein, 112 aa, Artificial Sequence
  Variable domain of HCD122 light chain
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 17 - protein, 219 aa, Artificial Sequence
  Variable and constant domains of HCD122 light chain
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 120 aa, Artificial Sequence
  Variable domain of HCD122 heavy chain
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 19 - protein, 450 aa, Artificial Sequence
  Variable and constant domains of HCD122 heavy chain
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPASKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 450 aa, Artificial Sequence
  Variable and constant domains of HCD122 heavy chain variant
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK